(19) United States
(12) Patent Application Publication
(10) Pub. No.: US 2010/0234716 A1
(43) Pub. Date: Sep. 16, 2010

(54) Method and Apparatus for Monitoring Fluid Content within Body Tissues

(75) Inventor: Jonathan Engel, Minneapolis, MN (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Corventis, Inc., St. Paul, MN (US)

(21) Appl. No.: 12/723,507

(22) Filed: Mar. 12, 2010

Related U.S. Application Data
(60) Provisional application No. 61/159,733, filed on Mar. 12, 2009.

(51) Int. Cl. A61B 8/14; A61B 5/04

(52) U.S. Cl. 600/391; 600/459

(57) ABSTRACT

Methods and devices for monitoring fluid content within body tissues. An adherent device having a support configured to transmit a signal into a body of a patient, and receive a reflected portion of the signal, and adhere to the skin of the patient. In many embodiments, the adherent device includes an ultrasonic transducer and other sensors. In many embodiments, the ultrasonic transducer is used in coordination with the other sensors to predict a cardiac decompensation.

### CROSS-REFERENCES TO RELATED APPLICATIONS

This application claims the benefit of U.S. Provisional Patent Application No. 61/159,733, filed on Mar. 12, 2009, the entirety of which is incorporated herein by reference for all reasons.

### BACKGROUND OF THE INVENTION

The present invention relates to patient monitoring. Although embodiments make specific reference to monitoring patients with an adherent patch device to detect fluid in the lungs, the system methods and device described herein may be applicable to many applications in which physiological monitoring is used, for example wireless physiological monitoring with devices for extended periods.

Patients are often treated for diseases and/or conditions associated with a compromised status of the patient, for example a compromised physiologic status such as heart disease. In some instances a patient may have suffered a heart attack and require care and/or monitoring after release from the hospital. While such long term care may be at least partially effective, many patients may not be sufficiently monitored and may eventually succumb to cardiac decompensation or heart failure. One example of a device that may be used to monitor a patient is the Holter monitor, or ambulatory electrocardiography device. Although such a device may be effective in measuring electrocardiography, such measurements alone may not be sufficient to reliably detect and/or avoid an impending cardiac decompensation.

Work in relation to embodiments of the present invention suggests that known methods and apparatus for long term monitoring of patients may be less than ideal to detect and/or avoid an impending cardiac decompensation. In at least some instances, cardiac decompensation can be difficult to detect, for example in the early stages. At least some of the known devices may not collect the right kinds of data to treat patients optimally. For example, although successful at detecting and storing electrocardiogram signals, devices such as the Holter monitor can be somewhat bulky and may not collect all of the kinds of data that would be ideal to diagnose and/or treat a patient, for example to detect decompensation.

Although the build-up of liquid in the patient may be a symptom of compromised physiologic status of the patient, at least some of the current methods and apparatus used to measure patient hydration may not be well suited and/or effective for long term monitoring of the patient. Although impedance may be used to measure patient hydration, at least some of the current methods of measuring patient hydration with impedance may be somewhat indirect and may be less than ideal to detect an impending patient decompensation. Although in hospital measurements based on tissue imaging may be used to measure hydration, for example tissue imaging with x-rays and ultrasound, such instrumentation can be complex and may not be well suited for the patient to use in his or her home in at least some instances. For example, the ultrasound and x-ray devices may be relatively large, expensive and complex, and may also require trained personnel for use in a dedicated care center in at least some instances. Because clinical signs related to increased tissue hydration such as distressed breathing may not be present until a substantial volume of liquid has accumulated in the lungs of the patient, at least some patients who have been released from the hospital may not seek care until the accumulated liquid has reached a significant and/or dangerous size in at least some instances. Also, patients who are in a hospital setting may not be properly diagnosed until an imaging study is completed, such that proper diagnoses of the patient in the hospital setting may not occur as quickly as would be ideal in at least some instances. Consequently, at least some patient may suffer from cardiac decompensation and may be subjected to more urgent care than would be ideal in at least some instances.

Therefore, a need exists for improved patient monitoring that overcomes at least some of the above mentioned disadvantages of the current detection devices. Ideally, such improved patient monitoring would provide a notification of increased tissue hydration before the tissue hydration becomes clinically noticeable by the patient. Also, it would be helpful if the monitoring was available to the patient outside a care center, for example available for in home monitoring.

### BRIEF SUMMARY OF THE INVENTION

The present invention relates to patient monitoring. Although embodiments make specific reference to monitoring patients with an adherent device comprising an ultrasound transducer, the systems, methods and device described herein may be applicable to many applications in which physiological monitoring is used, for example wireless physiological monitoring with devices for extended periods.

Embodiments of the present invention provide improved methods and devices for monitoring the fluid of body tissues, such as the lungs, with an adherent device. The adherent device comprises an ultrasonic transducer and at least one patient measurement sensor. The ultrasonic transducer can be configured to target a tissue of interest, for example lung tissue, and the at least one sensor can be coupled with the ultrasonic transducer to determine the fluid of the tissue, so as to improve the accuracy and reliability of the fluid determination. An amount of fluid of the tissue may be determined in many ways, and the amount may comprise a relative amount of fluid, for example based on a relative intensity of the ultrasound signal. In many embodiments, the hydration of the target tissue can be determined without imaging, which can benefit both in home monitoring in measurements in hospitals. The adherent device may comprise a processor coupled to the ultrasonic transducer and the at least one sensor to determine the fluid of the tissue. For example the processor can be configured to determine when the patient is positioned on a fluid sensitive orientation, and transmit the ultrasound signal in response to the fluid sensitive orientation. The ultrasonic transducer and at least one measurement sensor can be coupled to a support and configured to measure the patient data when the support is adhered to the patient. For example, the ultrasonic transducer and an electrocardiogram electrode can be positioned on the support so as to couple to the patient with a gel pad in contact with the electrode and the ultrasonic transducer.

In a first aspect, embodiments of the present invention provide a method for monitoring a patient having a skin and tissue disposed under the skin. The method comprises adhering an adherent device to the skin of the patient, the adherent device comprising an ultrasound transducer and at least one sensor configured to measure patient data, receiving an ultrasound signal reflected from the tissue, and determining an amount of fluid of the tissue based on the reflected signal and the patient data measured with the at least one sensor.

In many embodiments, the adherent device is adhered to the skin of the patient at a location corresponding to an accumulation of the fluid in the tissue when the patient is positioned in a fluid sensitive orientation and wherein the sensor measures the fluid sensitive orientation of the patient and the ultrasound signal is transmitted in response to the fluid sensitive orientation of the patient.

In many embodiments, the amount of fluid is determined in response to a tissue penetration depth of the ultrasound signal.

In many embodiments, the amount of fluid is determined without imaging the tissue.

In many embodiments, the method further comprises transmitting the acoustic signal into the patient from the adherent device adhered to the skin.

In many embodiments, the fluid comprises a liquid.

In many embodiments, the amount of fluid comprises at least one of a percentage of fluid of the tissue, a relative amount of fluid, a change from a baseline or a percent change of a peak of the signal reflected from the tissue over time.

In many embodiments, the sensor comprises a plurality of sensors coupled to a support of the adherent device, the plurality of sensors configured measure patient information selected from a group consisting of: ECG, tissue resistance, bioimpedance, respiration, respiration rate variability, heart rate, heart rhythm, heart rate variability, heart rate turbulence, heart sounds, respiratory sounds, blood pressure, activity, posture, wake, sleep, orthopnea, temperature, heat flux, and weight.

In many embodiments, the signal is configured to measure the amount of fluid from a lung of the patient to determine an amount of fluid disposed in the pleura of the lung.

In many embodiments, the signal is configured to measure the amount of fluid from a lung of the patient to determine an amount of fluid disposed within lung tissue of the lung.

In many embodiments, the signal is reflected of a portion of a lung of the patient.

In many embodiments, the reflected portion of the signal is received by an ultrasonic transducer of the adherent device, which also transmits the signal.

In many embodiments, determining the fluid amount disposed in the tissue comprises determining a fluid disposed within a portion of a lung of the patient. In many embodiments, determining a fluid disposed within the portion of the lung comprises comparing a first reflected portion of the signal with a second reflected portion of the signal. In many embodiments, determining the patient's risk of a cardiac decompensation based on the fluid content within the portion of the lung. In many embodiments, the method further comprises verifying the patient's risk of cardiac decompensation based on the sensor information.

In many embodiments, the method further comprises determining the patient's breathing cycles using the adherent device and coordinating transmitting the signal based on the breathing cycles.

In many embodiments, the method further comprises determining the patient's sleep status using the adherent device and coordinating transmitting the signal based on the sleep status.

In many embodiments, the method further comprises determining an orientation of the patient using the adherent device and transmitting the signal based on the determined orientation.

In many embodiments, the transmitting the signal occurs after determining that the orientation of the patient is in a standing or upright position. In many embodiments, the adherent device includes an accelerometer to determine the orientation of the patient. In many embodiments, the accelerometer comprises at least one measurement axis sensitive to gravity aligned with an electrode measurement axis which extends along the adherent device.

In many embodiments, the method further comprises determining the patient's heart rate using the adherent device and coordinating transmitting the signal based on the heart rate.

In many embodiments, the method comprises transmitting a wireless signal to an external device based on determining the fluid content in the body.

In many embodiments, an electrically conductive gel pad is coupled to the skin of the patient when the device is adhered to the skin, and the ultrasound signal is received through the electrically conductive gel pad.

In another aspect, embodiments of the present invention provide an adherent device to measure data from a patient having skin and a tissue disposed beneath the skin. The device comprising at least one ultrasonic transducer, at least one sensor configured to measure patient data, a support configured to adhere to the skin of the patient to couple the ultrasonic transducer and the at least one sensor to the skin, and circuitry supported with the support and coupled the ultrasonic transducer and the sensor, the circuitry configured to receive an ultrasound signal reflected from the tissue and the patient data from the at least one sensor.

In many embodiments, the support comprises a flexible support configured to stretch with a skin of the patient.

In many embodiments, the at least one ultrasonic transducer comprises a piezoelectric ceramic material.

In many embodiments, the at least one ultrasonic transducer comprises an array of transducers.

In many embodiments, the at least one transducer is configured to measure a lung of the patient to determine an amount of fluid present in a pleura of the lung.

In many embodiments, the ultrasonic transducer is configured to receive a reflected portion of the signal.

In many embodiments, the support further comprises a second ultrasonic transducer which is configured to receive a reflected portion of the signal.

In many embodiments, the signal is reflected from a portion of a lung of the patient.

In many embodiments, the at least one sensor comprises an electrode and a gel pad, wherein the ultrasonic transducer and the electrode are positioned on the support to couple the electrode and the ultrasonic transducer to the tissue with the gel pad when the support is adhered to the skin.

In many embodiments, the flexible support comprises a breathable tape with an adhesive coating, at least one electrode coupled to the breathable tape and capable of electrically coupling to a skin of the patient, a printed circuit board connected to the breathable tape to support the printed circuit board with the breathable tape when the tape is adhered to the patient; and electronic components electrically connected to the printed circuit board and coupled to the at least one electrode to measure physiologic signals of the patient, and coupled to the at least one ultrasonic transducer.

In many embodiments, the electronic components are coupled to the at least one sensor to measure the patient from a group consisting of: ECG, tissue resistance, bioimpedance, respiration, respiration rate variability, heart rate, heart rhythm, heart rate variability, heart rate turbulence, heart sounds, respiratory sounds, blood pressure, activity, posture, wake, sleep, orthopnea, temperature, heat flux, and weight.

In many embodiments, the activity sensor is chosen from a group consisting of: ball switch, accelerometer, minute ventilation, bioimpedance noise, muscle noise, and posture.

In many embodiments, the accelerometer comprises at least one measurement axis sensitive to gravity aligned with an electrode measurement axis which extends along the adherent device.

In many embodiments, the at least one sensor comprises a second electrode affixed to the breathable tape and capable of electrically coupling to a skin of the patient, and wherein the electronic components comprise an accelerometer and a processor, and wherein the electrodes are separated by a distance to define an electrode measurement axis, and wherein the processor determines an orientation of the electrode measurement axis on the patient in response to an accelerometer signal.

In many embodiments, the at least one electrode and second electrode comprise a positive and negative electrode that define an orientation of an electrode measurement vector along the electrode measurement axis.

In many embodiments, the support further comprises at least one gel pad attached to the breathable tape and coupled to the at least one electrode and the at least one ultrasonic transducer.

In many embodiments, the flexible support further comprises a gel cover coupled to the breathable tape opposite to the adhesive coating.

In many embodiments, the transducer is coupled to the gel cover.

In many embodiments, the flexible support further comprises a gel pad coupled to the adhesive coating, a portion of the gel pad extending into an opening of the breathable tape, the portion coupled to the gel cover.

In many embodiments, the electronic components comprise a processor configured to control the ultrasonic transducer. In many embodiments, the processor is further configured to control the ultrasonic transducer to receive a reflected portion of the ultrasonic signal. In many embodiments, the processor is further configured to determine a fluid disposed within a portion of a lung of the patient based on the reflected portion of the ultrasonic signal. In many embodiments, the processor is configured to determine a fluid disposed within the portion of the lung based on a first reflected portion of the signal compared with a second reflected portion of the signal.

In many embodiments, the processor comprises a processor system configured to determine the patient's risk of cardiac decompensation based on the reflected portion of the ultrasonic signal. In many embodiments, the electric components are coupled to the at least one sensor and the at least one electrode to provide physiological information of the patient to the processor, and the processor system is configured to verify the patient's risk of cardiac decompensation based on the physiological information.

In many embodiments, the processor is configured to control the ultrasonic transducer to transmit the ultrasonic signal at predetermined intervals.

In many embodiments, the electric components are coupled to the at least one sensor and the at least one electrode, and the processor is configured to determine breathing cycles of the patient using the at least one sensor, and to transmit the ultrasonic signal based on the breathing cycles.

In many embodiments, the electric components are coupled to at least one sensor and the at least one electrode, and the processor is further configured to determine a sleep status of the patient using the at least one sensor, and to transmit the ultrasonic signal based on the sleep status.

In many embodiments, the electric components are coupled to the at least one sensor and the at least one electrode, and the processor is further configured to determine an orientation of the patient using the at least one sensor, and to transmit the ultrasonic signal based on the orientation.

In many embodiments, the electric components are coupled to at least one sensor and the at least one electrode, and the processor is configured to determine a heart rate of the patient using the at least one sensor, and to transmit the ultrasonic signal based on the heart rate.

In many embodiments, the processor is configured to determine when the patient is positioned in a fluid sensitive orientation and the processor is configured to transmit the ultrasound signal in response to the fluid sensitive orientation.

In many embodiments, the processor is configured to determine the amount of fluid in response to the tissue penetration depth.

In another aspect, embodiments of the present invention comprise a method of determining hydration of a tissue of a patient having a skin. An ultrasound is transmitted signal through the skin, and the hydration of the tissue is determined in response to at least one of a return time or a penetration depth of the ultrasound signal.

In another aspect, embodiments of the present invention provide an device to determine hydration of a tissue of a patient having a skin. An ultrasound transducer is configured to transmit an ultrasound signal through the skin. A processor is coupled to the transducer and comprises a tangible medium configured to determine the hydration of the tissue in response to at least one of a return time or a tissue penetration depth of the ultrasound signal.

### BRIEF DESCRIPTION OF THE DRAWINGS

FIG. 1A shows a patient and a monitoring system comprising an adherent device configured to measure patient hydration with ultrasound, according to embodiments of the present invention;

FIG. 1B shows a bottom view of the adherent device as in FIG. 1A comprising an adherent patch;

FIG. 1C shows a top view of the adherent patch, as in FIG. 1B;

FIG. 1D shows a printed circuit boards and electronic components over the adherent patch, as in FIG. 1C;

FIG. 1D1 shows an equivalent circuit that can be used to determine optimal frequencies for determining patient hydration, according to embodiments of the present invention;

FIG. 1D2 shows an adherent devices as in FIGS. 1A-1D positioned on a patient to determine orientation of the adherent patch on the patient, according to embodiments of the present invention;

FIG. 1D3 shows vectors from a 3D accelerometer to determine orientation of the measurement axis of the patch adhered on the patient, according to embodiments of the present invention;

FIG. 1E shows batteries positioned over the printed circuit board and electronic components as in FIG. 1D;

FIG. 1F shows a top view of an electronics housing and a breathable cover over the batteries, electronic components and printed circuit board as in FIG. 1E;

FIG. 1G shows a side view of the adherent device as in FIGS. 1A to 1F;

FIG. 1H shows a bottom perspective view of the adherent device as in FIGS. 1A to 1G;

FIGS. 1I, 1IA, and 1J show a side cross-sectional view and an exploded view, respectively, of the adherent device as in FIGS. 1A to 1H;

FIGS. 1I1, and 1J1 show a side cross-sectional view and an exploded view, respectively, of embodiments of the adherent device with a temperature sensor affixed to the gel cover;

FIG. 1K shows at least one electrode configured to electrically couple to a skin of the patient through a breathable tape, according to embodiments of the present invention;

FIG. 2A shows a method of monitoring a patient, according to embodiments of the present invention;

FIGS. 3A and 3B shows cross-sectional views of an adherent device detecting a pleural effusion, according to embodiments of the present invention;

FIGS. 3C and 3D show graphical outputs of an adherent device as in FIGS. 3A and 3B; and

FIG. 4A shows a method for determining the a cardiac decompensation based on detecting a pleural effusion, according to embodiments of the present invention.

### DETAILED DESCRIPTION OF THE INVENTION

Embodiments of the present invention relate to patient monitoring. Although embodiments make specific reference to monitoring fluid content and electrocardiogram signals with an adherent patch, the system methods and device described herein may be applicable to any application in which physiological monitoring is used, for example wireless physiological monitoring for extended periods.

Embodiments of the present invention provide methods and devices for monitoring fluid content within body tissues, such as the lungs, via impingement of ultrasonic signals into the body through an adherent device and by analysis of a return signal. Changes in fluid content can manifest as altered return signals when compared to data collected over the life of the adherent device or a series of adherent devices. Differences in ultrasound propagation may be used to determine fluid content in body tissues, and to monitor fluid content, and thus monitor the progress of various clinical conditions. The difference in hydration over time can be determined in many ways. For example, in the lungs a large acoustic impedance mismatch between the air spaces in the lungs, and the surrounding tissue and fluid, such that the depth and quality of an ultrasonic return signal incident on the lungs can change dramatically as the fluid content of the lungs changes, for example as occurs in some types of heart failure. Thus, many embodiments use an ultrasonic transducer of an adherent device to monitor over time the depth and characteristics of ultrasonic return signals of underlying tissues to track and predict clinical events such as cardiac decompensation. An associated signal processing circuitry may comprise a microcontroller and a digital signal processor, which circuitry can record and process the ultrasonic return data to extract features of interest and transmit and store this information as needed to determine patient hydration.

The change in hydration can be determined in many ways with the ultrasound transducer and associated circuitry so as to target hydration of tissues of interest, for example tissues related to the lung of the patient. For example, the patient may be asked to inhale and exhale, and a plurality of signals measured to determine a profile of the ultrasound return signal over time as the patient inhales and exhales. The magnitude of the change in the reflected signal can be related to the hydration and air capacity of the lungs.

Measurements from at least one additional sensor from the adherent device can be coupled with the ultrasound measurement. For example, an accelerometer can be used to determine the orientation of the patient when the ultrasound hydration measurement is made. A series of measurements can be taken at night when the patient lays prone, and several measurements taken over the course of at least one week, for example one month.

The adherent devices as described herein can be positioned on the thorax of the patient to detect the accumulation of fluid, for example pleural fluid and/or fluid within the lung tissue, such that the accumulation can be detected with in home monitoring so as to allow for early intervention that can minimize, even avoid, hospitalization and/or ICU care. The pleura is a membrane covering the lungs, and an over accumulation of pleural fluid can be referred to as a pleural effusion. The pleura includes an inner layer, visceral pleura VP, which covers the parenchyma of the lungs, and an outer layer, parietal pleura PP, which covers the chest wall and diaphragm. Normally a very small amount of pleural fluid exists in the pleural space between the visceral pleura and parietal pleura. The pleural space normally has thickness of 10-20 μm between the visceral pleura and parietal pleura, and sometimes no space is readably discernable.

Although fluid corresponding to the pleural space can be measured to determine hydration, work in relation to embodiments of the present invention suggests that the ultrasound signal from the lung away from the pleural space may be measured to determine hydration. For example, increased expansion of the pleural space may result in a slight compression of the lung such that the alveoli of the lung have a slightly smaller volume resulting in a decrease in an intensity of the reflected signal.

Fluid can also build up in lung tissue as a result of a pulmonary edema. A pulmonary edema is the abnormal presence of fluid in the parenchyma comprising alveoli (air sacs) of the lungs, which may result from congestive heart failure. When the left ventricle begins to fail, pressure increases inside the left atrium and the pulmonary veins and capillaries. The pressure increase causes fluid to be pushed through the capillary walls into the air sacs of the lungs. Symptoms of a pulmonary edema can occur suddenly or slowly develop over weeks. Pulmonary edema can be life-threatening to the patient.

The adherent device comprises a support, for example a patch that may comprise breathable tape, and the support can be configured to adhere to the patient and support the electronics and sensors on the patient. The support can be porous and breathable so as to allow water vapor transmission. The support can also stretch with skin of the patient, so as to improve patient comfort and extend the time that the support can be adhered to the patient.

Decompensation is failure of the heart to maintain adequate blood circulation. Although the heart can maintain at least many pumping of blood, the quantity is inadequate to maintain healthy tissues. Several symptoms can result from decompensation including pulmonary congestion, breathlessness, faintness, cardiac palpitation, edema of the extremities, and enlargement of the liver. Cardiac decompensation can result in slow or sudden death. Sudden Cardiac Arrest (hereinafter “SCA”), also referred to as sudden cardiac death, is an abrupt loss of cardiac pumping function that can be caused by a ventricular arrhythmia, for example ventricular tachycardia and/or ventricular fibrillation. Although decompensation and SCA can be related in that patients with decompensation are also at an increased risk for SCA, decompensation is primarily a mechanical dysfunction caused by inadequate blood flow, and SCA is primarily an electrical dysfunction caused by inadequate and/or inappropriate electrical signals of the heart.

In many embodiments, the adherent devices described herein may be used for 90 day monitoring, or more, and may comprise completely disposable components and/or reusable components, and can provide reliable data acquisition and transfer. In many embodiments, the patch is configured for patient comfort, such that the adherent patch can be worn and/or tolerated by the patient for extended periods, for example 90 days or more. The patch may be worn continuously for at least seven days, for example 14 days, and then replaced with another patch. In many embodiments, the adherent patch comprises a tape, which comprises a material, preferably breathable, with an adhesive, such that trauma to the patient skin can be minimized while the patch is worn for the extended period. The printed circuit board may comprise a flex printed circuit board that can flex with the patient to provide improved patient comfort.

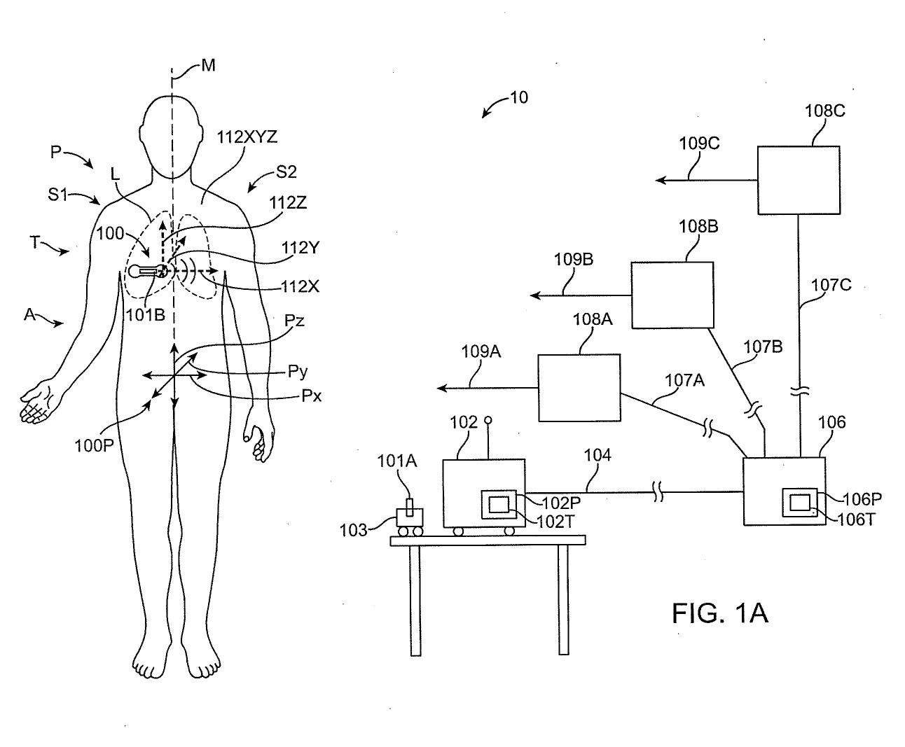

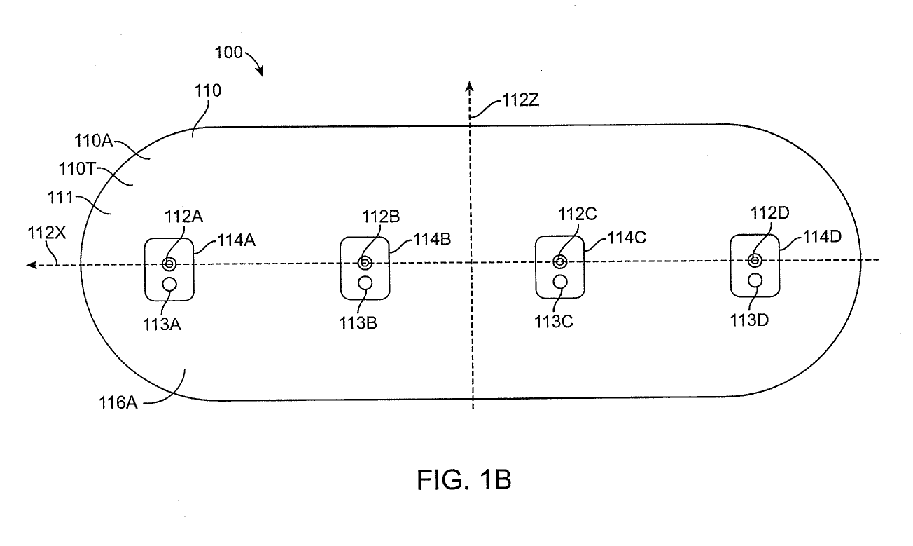

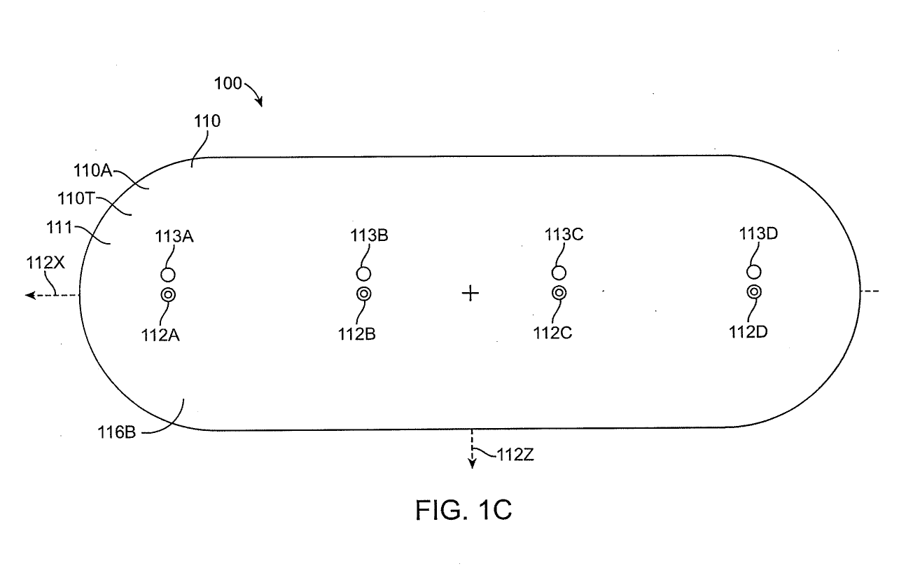

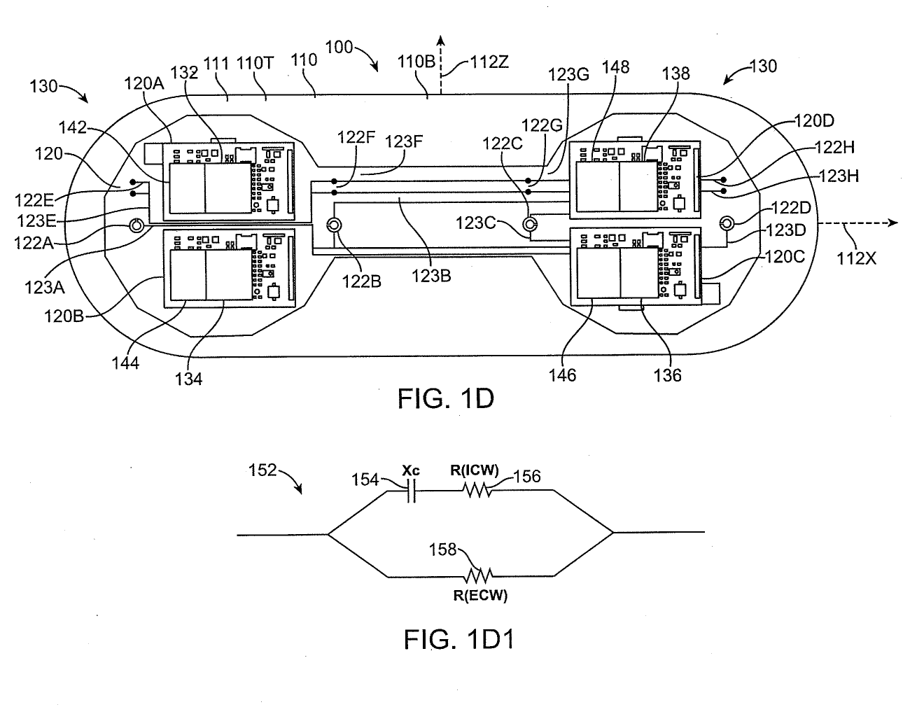

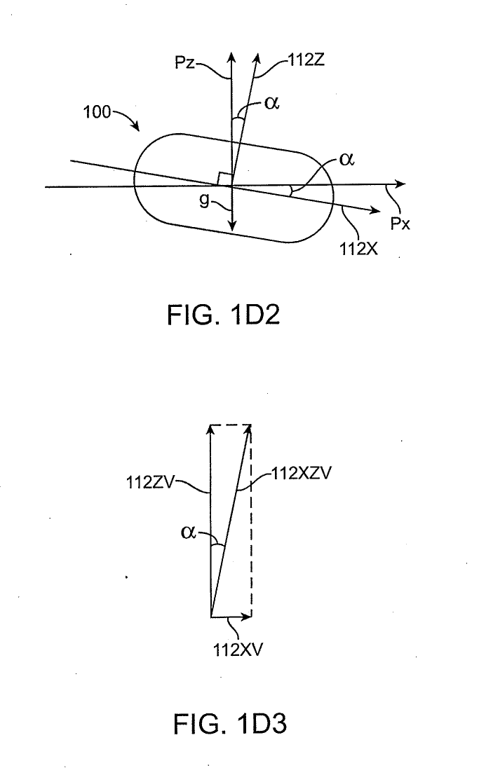

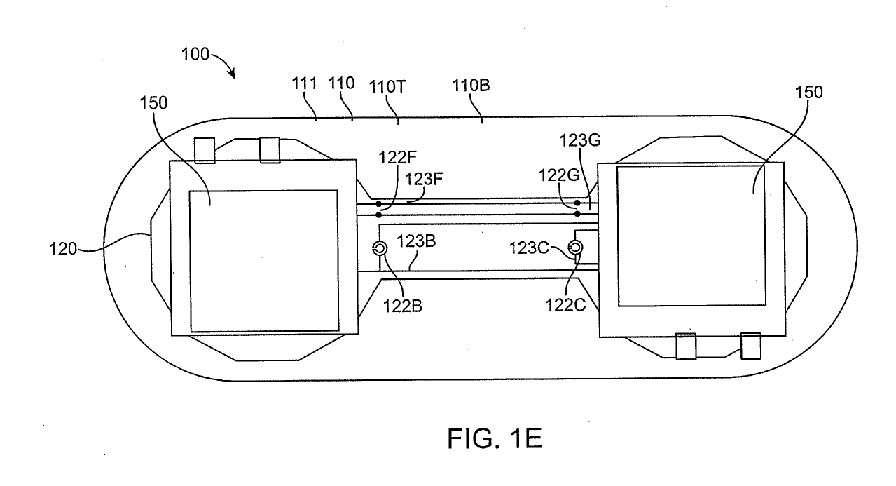

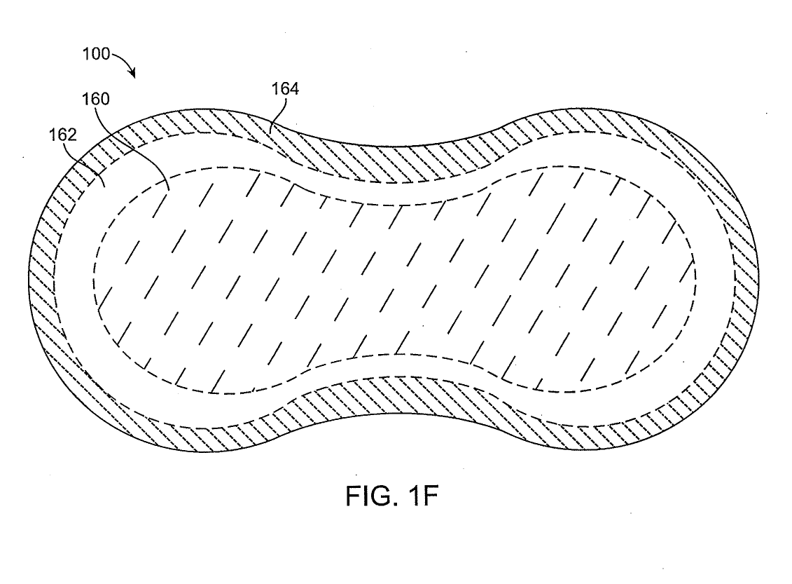

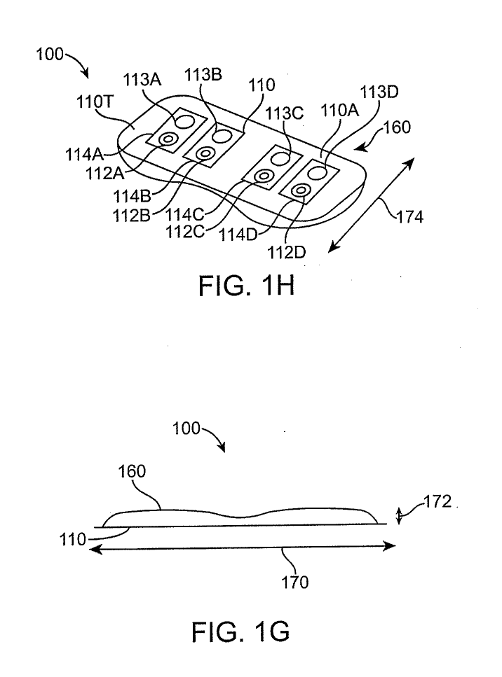

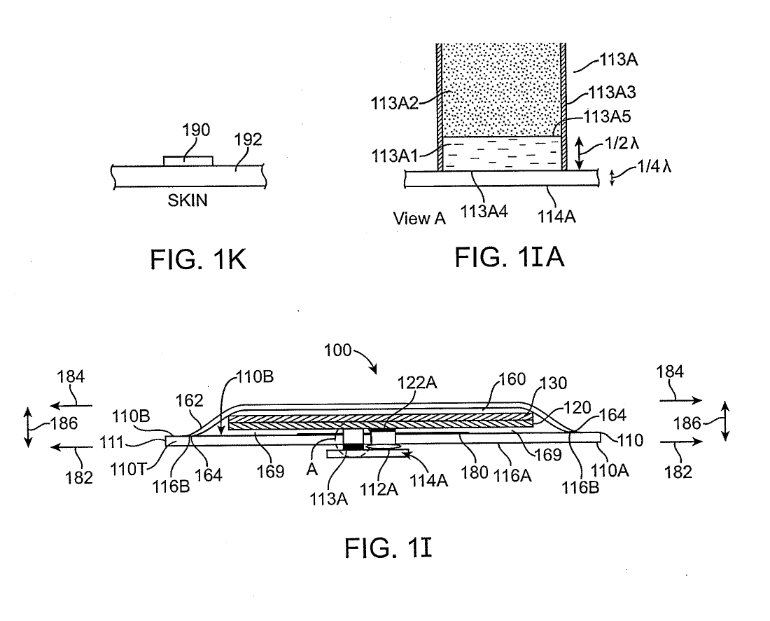

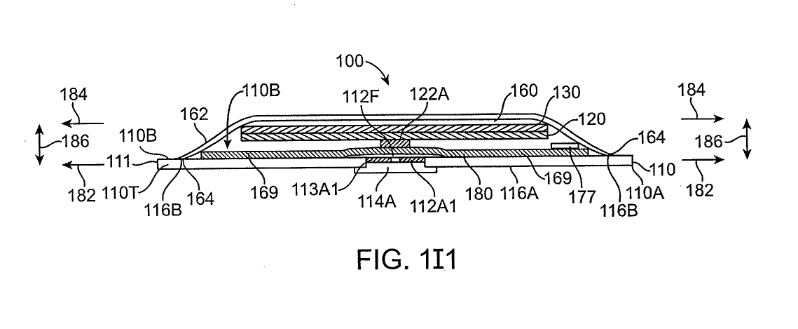

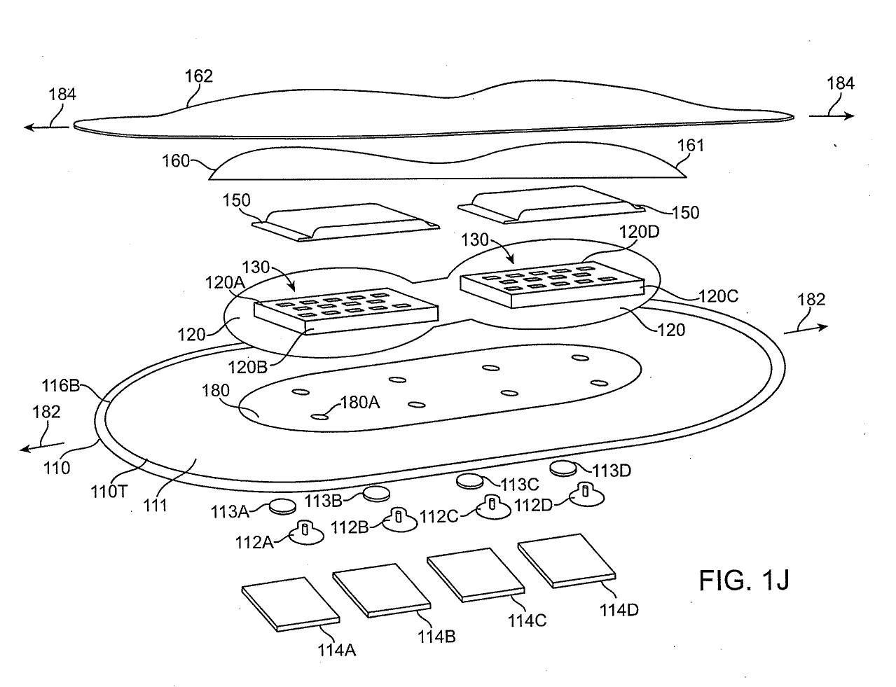

FIG. 1A shows a patient P and a monitoring system 10, in which the monitoring system comprises an adherent device configured to measure patient hydration with ultrasound. Patient P comprises a midline M, a first side S1, for example a right side, and a second side S2, for example a left side. Monitoring system 10 comprises an adherent device 100. Adherent device 100 can be adhered to a patient P at many locations, for example thorax T of patient P, and directly external to the lungs L of the patient P. The adherent device may be placed on the thorax of the patient so as to target selectively the tissues of lung L for hydration measurement with the ultrasound energy. In many embodiments, the adherent device may adhere to one side of the patient, from which side data can be collected. Work in relation with embodiments of the present invention suggests that location on a side of the patient can provide comfort for the patient while the device is adhered to the patient.

Adherent device 100 can be aligned and/or oriented with respect to axes of patient P. Orientation of adherent device 100 can comprise orientation of device 100 with a patient coordinate system 100P aligned with axes of the patient. Patient P comprises a horizontal axis Px that extends laterally from one side of the patient to the other, for example from side S1 to side S1 across midline M. Patient P comprises an anterior posterior axis Py that extends from the front, or anterior, of the patient to the back, or posterior of the patient. Patient P comprises a vertical axis Pz that extends vertically along the patient, for example vertically along the midline of the patient from the feet of the patient toward the head of the patient. In many embodiments, horizontal axis Px, anterior posterior axis Py and vertical axis Pz may comprise a right handed triple of orthogonal coordinate references.

Adherent device 100 may comprise a 3D coordinate reference system 112XYZ. Device 100 may comprise an X-axis 112X for alignment with horizontal axis Px of the patient, a Y-axis for alignment with anterior posterior axis Py of the patient and a Z axis for alignment with vertical axis Pz of the patient. Coordinate reference system 112XYZ may comprise X-axis 112X, Y-axis 112Y and Z-axis 112Z. Coordinate reference system 112XYZ may comprise a right handed triple, although other non-orthogonal and orthogonal reference systems may be used.

Adherent device 100 may comprise indicia for alignment with an axis of the patient. The indicia can be used to align at least one axis of device 100 with at least one axis of the patient. The indicia can be positioned on at least one of the adherent patch, a cover, or an electronics module. The indicia can be visible to the patient and/or a care provider to adhere device 100 to the patient in alignment with at least one axis of the patient. A vertical line along Z-axis 112Z can indicate vertical axis 112Z to the patient and/or care provider, and a horizontal line along X-axis 112X can indicate horizontal X-axis 112X to the patient and/or care provider. A name, logo and/or trademark can be visible the outside of device 100 to indicate that device 100 correctly oriented, and arrows can also be used, for example a vertical arrow pointing up and a horizontal arrow pointing to the right.

Monitoring system 10 includes components to transmit data to a remote center 106. Remote center 106 can be located in a different building from the patient, for example in the same town as the patient, and can be located as far from the patient as a separate continent from the patient, for example the patient located on a first continent and the remote center located on a second continent. Adherent device 100 can communicate wirelessly to an intermediate device 102, for example with a single wireless hop from the adherent device on the patient to the intermediate device. Intermediate device 102 can communicate with remote center 106 in many ways, for example with an internet connection and/or with a cellular connection. In many embodiments, monitoring system 10 comprises a distributed processing system with at least one processor comprising a tangible medium of device 100, at least one processor 102P of intermediate device 102, and at least one processor 106P at remote center 106, each of which processors can be in electronic communication with the other processors. At least one processor 102P comprises a tangible medium 102T, and at least one processor 106P comprises a tangible medium 106T. Remote processor 106P may comprise a backend server located at the remote center. Remote center 106 can be in communication with a health care provider 108A with a communication system 107A, such as the Internet, an intranet, phone lines, wireless and/or satellite phone. Health care provider 108A, for example a family member, can be in communication with patient P with a communication, for example with a two way communication system, as indicated by arrow 109A, for example by cell phone, email, landline. Remote center 106 can be in communication with a health care professional, for example a physician 108B, with a communication system 107B, such as the Internet, an intranet, phone lines, wireless and/or satellite phone. Physician 108B can be in communication with patient P with a communication, for example with a two way communication system, as indicated by arrow 109B, for example by cell phone, email, landline. Remote center 106 can be in communication with an emergency responder 108C, for example a 911 operator and/or paramedic, with a communication system 107C, such as the Internet, an intranet, phone lines, wireless and/or satellite phone. Emergency responder 108C can travel to the patient as indicated by arrow 109C. Thus, in many embodiments, monitoring system 10 comprises a closed loop system in which patient care can be monitored and implemented from the remote center in response to signals from the adherent device.

In many embodiments, the adherent device may continuously monitor physiological parameters, communicate wirelessly with a remote center, and provide alerts when necessary. The system may comprise an adherent patch, which attaches to the patient's thorax and contains sensing electrodes, battery, memory, logic, and wireless communication capabilities. In many embodiments, the patch can communicate with the remote center, via the intermediate device in the patient's home. In many embodiments, remote center 106 receives the patient data and applies a patient evaluation algorithm, for example the prediction algorithm to predict cardiac decompensation. In many embodiments, the algorithm may comprise an algorithm to predict impending cardiac decompensation. When a flag is raised, the center may communicate with the patient, hospital, nurse, and/or physician to allow for therapeutic intervention, for example to prevent decompensation.

The adherent device may be affixed and/or adhered to the body in many ways. For example, with at least one of the following an adhesive tape, a constant-force spring, suspenders around shoulders, a screw-in microneedle electrode, a pre-shaped electronics module to shape fabric to a thorax, a pinch onto roll of skin, or transcutaneous anchoring. Patch and/or device replacement may occur with a keyed patch (e.g. two-part patch), an outline or anatomical mark, a low-adhesive guide (place guide|remove old patch|place new patch|remove guide), or a keyed attachment for chatter reduction. The patch and/or device may comprise an adhesiveless embodiment (e.g. chest strap), and/or a low-irritation adhesive for sensitive skin. The adherent patch and/or device can comprise many shapes, for example at least one of a dogbone, an hourglass, an oblong, a circular or an oval shape.

In many embodiments, the adherent device may comprise a reusable electronics module with replaceable patches, and each of the replaceable patches may include a battery. The module may collect cumulative data for approximately 90 days and/or the entire adherent component (electronics+patch) may be disposable. In a completely disposable embodiment, a “baton” mechanism may be used for data transfer and retention, for example baton transfer may include baseline information. In many embodiments, the device may have a rechargeable module, and may use dual battery and/or electronics modules, wherein one module 101A can be recharged using a charging station 103 while the other module 101B is placed on the adherent patch with connectors. In many embodiments, the intermediate device 102 may comprise the charging module, data transfer, storage and/or transmission, such that one of the electronics modules can be placed in the intermediate device for charging and/or data transfer while the other electronics module is worn by the patient.

System 10 can perform the following functions: initiation, programming, measuring, storing, analyzing, communicating, predicting, and displaying. The adherent device may contain a subset of the following physiological sensors: bioimpedance, respiration, respiration rate variability, heart rate (ave, min, max), heart rhythm, hear rate variability (HRV), heart rate turbulence (HRT), heart sounds (e.g. S3), respiratory sounds, blood pressure, activity, posture, wake/sleep, orthopnea, temperature/heat flux, and weight. The activity sensor may comprise one or more of the following: ball switch, accelerometer, minute ventilation, HR, bioimpedance noise, skin temperature/heat flux, BP, muscle noise, posture.

The adherent device can wirelessly communicate with remote center 106. The communication may occur directly (via a cellular or Wi-Fi network), or indirectly through intermediate device 102. Intermediate device 102 may consist of multiple devices, which can communicate wired or wirelessly to relay data to remote center 106.

In many embodiments, instructions are transmitted from remote site 106 to a processor supported with the adherent patch on the patient, and the processor supported with the patient can receive updated instructions for the patient treatment and/or monitoring, for example while worn by the patient.

FIG. 1B shows a bottom view of adherent device 100 as in FIG. 1A comprising an adherent patch 110. Adherent patch 110 comprises a first side, or a lower side 110A, that is oriented toward the skin of the patient when placed on the patient. In many embodiments, adherent patch 110 comprises a tape 110T which is a material, preferably breathable, with an adhesive 116A. Patient side 110A comprises adhesive 116A to adhere the patch 110 and adherent device 100 to patient P. Electrodes 112A, 112B, 112C and 112D are affixed to adherent patch 110. In many embodiments, at least four electrodes are attached to the patch, for example six electrodes. In many embodiments the patch comprises two electrodes, for example two electrodes to measure the electrocardiogram (ECG) of the patient. Gel 114A, gel 114B, gel 114C and gel 114D can each be positioned over electrodes 112A-D, respectively, to provide electrical conductivity between the electrodes and the skin of the patient. In many embodiments, the electrodes can be affixed to the patch 110, for example with known methods and structures such as rivets, adhesive, stitches, etc. Ultrasonic transducers 113A, 113B, 113C, and 113D are also affixed to adherent patch 110, adjacent to the electrodes 112A-D. Gels 114A-D can each be positioned over ultrasonic transducers 113A-D, respectively, to provide acoustic conductivity between the ultrasonic transducers 113A-D and the skin of the patient. Gels 114A-D can be acoustically impedance matched to the ultrasonic transducers 113A-D such that the thickness of each gel 114A-D is a ¼ wavelength thick with respect to the center wavelength of the ultrasonic transducers 113A-D, or equally divisible by ¼ of a center wavelength of each ultrasonic transducer 113A-D. In many embodiments the ultrasonic transducers 113A-D do not include gel layers and are configured to directly couple to skin, where acoustic coupling occurs from oil or sweat production in the skin. In many embodiments only one ultrasonic transducer is used. In many embodiments, two ultrasonic transducers are used. In many embodiments, more than four ultrasonic transducers may be used. In many embodiments, patch 110 comprises a breathable material to permit air and/or vapor to flow to and from the surface of the skin.

Electrodes 112A-D extend substantially along a horizontal measurement axis that corresponds to X axis-112X of the measurement device. Electrodes 112-D can be affixed to adherent patch 110A, such that the positions of electrodes 112A-D comprise predetermined positions on adherent patch 110A. Z-axis 112Z can extend perpendicular to the electrode measurement axis, for example vertically and perpendicular to X-axis 112 when adhered on the patient. X-axis 112× and Z-axis 112Z can extend along an adhesive surface of adherent patch 110A, and a Y-axis 112Y can extend away from the adhesive surface of adherent device 110A.

FIG. 1C shows a top view of the adherent patch 100, as in FIG. 1B. Adherent patch 100 comprises a second side, or upper side 110B. In many embodiments, electrodes 112A-D and ultrasonic transducers 113A-D extend from lower side 110A through adherent patch 110 to upper side 110B. An adhesive 116B can be applied to upper side 110E to adhere structures, for example a breathable cover, to the patch such that the patch can support the electronics and other structures when the patch is adhered to the patient. The PCB may comprise completely flex PCB, rigid PCB, rigid PCB combined flex PCB and/or rigid PCB boards connected by cable.

FIG. 1D shows a printed circuit boards and electronic components over adherent patch 110, as in FIGS. 1A to 1C. In many embodiments, a printed circuit board (PCB), for example flex printed circuit board 120, may be connected to electrodes 112A-D with connectors 122A, 122B, 122C and 122D. In many embodiments, a printed circuit board (PCB), for example flex printed circuit board 120, may be connected to electrodes 112A-D with connectors 122A-D. Flex printed circuit board 120 can include traces 123A, 123B, 123C and 123D that extend to connectors 122A-D, respectively, on the flex PCB. Connectors 122A-D can be positioned on flex printed circuit board 120 in alignment with electrodes 112A-D so as to electrically couple the flex PCB with the electrodes. In many embodiments, the printed circuit board (PCB), for example the flex printed circuit board 120, may be connected to ultrasonic transducers 113A-D with connectors 122E, 122F, 122G and 122H. Flex printed circuit board 120 can include traces 123E-H that extend to connectors 122E-H, respectively, on the flex PCB. Connectors 122E-H can be positioned on flex printed circuit board 120 in alignment with ultrasonic transducers 113A-D so as to electrically couple the flex PCB with the ultrasonic transducers. In many embodiments, connectors 122A-H may comprise insulated wires and/or a film with conductive ink that provide strain relief between the PCB and the electrodes, and ultrasonic transducers. For example, connectors 122A-H may comprise a flexible film, such as at least one of known polyester film or known polyurethane file coated with a conductive ink, for example a conductive silver ink. In many embodiments, additional PCB's, for example rigid PCB's 120A, 120B, 120C and 120D, can be connected to flex printed circuit board 120. Electronic components 130 can be connected to flex printed circuit board 120 and/or mounted thereon. In many embodiments, electronic components 130 can be mounted on the additional PCB's.

Electronic components 130 comprise components to take physiologic measurements, transmit data to remote center 106 and receive commands from remote center 106. In many embodiments, electronics components 130 may comprise known low power circuitry, for example complementary metal oxide semiconductor (CMOS) circuitry components. Electronics components 130 comprise an activity sensor and activity circuitry 134, impedance circuitry 136 and electrocardiogram circuitry, for example ECG circuitry 136. In many embodiments, electronics circuitry 130 may comprise a microphone and microphone circuitry 142 to detect an audio signal from within the patient, and the audio signal may comprise a heart sound and/or a respiratory sound, for example an S3 heart sound and a respiratory sound with rales and/or crackles. In many embodiments electronic components comprise circuitry and at least one sensor to measure at least one of ECG, tissue resistance, bioimpedance, respiration, respiration rate variability, heart rate, heart rhythm, heart rate variability, heart rate turbulence, heart sounds, respiratory sounds, blood pressure, activity, posture, wake, sleep, orthopnea or temperature and heat flux.

Electronics circuitry 130 may comprise a temperature sensor, for example a thermistor in contact with the skin of the patient, and temperature sensor circuitry 144 to measure a temperature of the patient, for example a temperature of the skin of the patient. A temperature sensor may be used to determine the sleep and wake state of the patient. The temperature of the patient can decrease as the patient goes to sleep and increase when the patient wakes up.

Electronics circuitry 130 may comprise piezoelectric driving circuitry coupled to ultrasonic transducers 113A-D. The piezoelectric driving circuitry may include transmitting, amplification, voltage transformation, time-gain decompensation, signal processing, analog to digital conversion, and capacitive circuitry. The piezoelectric driving may be configured to provide energy to the ultrasonic transducers 113A-D such that the ultrasonic transducers generate signals ranging from 1 to 20 MHz. The piezoelectric driving circuitry may be configured to process reflected ultrasonic signals which are received by the ultrasonic transducers 113A-D. In many embodiments the piezoelectric driving circuitry may be configured to determine a distance from the ultrasonic transducers 113A-D to one or more reflective surfaces which reflect ultrasonic signals. In many embodiments the piezoelectric driving circuitry is configured to characterize reflected ultrasonic signals received by the ultrasonic transducers 113A-D as a fluid content signal. In many embodiments the fluid content signal is an indication of fluid content in a portion of a patient's body. In many embodiments the fluid content signal is an indication of fluid content in a portion of the pleura of a patient's lung. In many embodiments the piezoelectric driving circuitry may be configured to provide a plurality of different power outputs to the ultrasonic transducers 113A-D, such that each ultrasonic transducer transmits at different frequencies. In many embodiments the piezoelectric driving circuitry is configured to provide pulsed power to the ultrasonic transducers 113A-D, such that the ultrasonic transducers 113A-D transmit ultrasonic signals at predetermined intervals. In many embodiments the piezoelectric driving circuitry is configured to provide power to the ultrasonic transducers 113A-D such that the ultrasonic transducers 113A-D transmit ultrasonic signals. In many embodiments the piezoelectric driving circuitry is configured to provide a varying spectrum of power to the ultrasonic transducers 113A-D. In many embodiments the piezoelectric driving circuitry is configured to operate the ultrasonic transducers 113A-D as a pulsed and/or phased array. In many embodiments the piezoelectric driving circuitry 113A-D is configured to provide power to only some of the ultrasonic transducers 113A-D, and process reflected ultrasonic signals from only some of the ultrasonic transducers 113A-D. In many embodiments the piezoelectric driving circuitry is configured to operate the ultrasonic transducers 113A-D in a continuous or pulsed Doppler mode. In many embodiments the piezoelectric driving circuitry is configured to output information from the ultrasonic transducers 113A-D in a-mode (amplitude), b-mode (brightness), or m-mode (motion) to an external or internal visual device.

Work in relation to embodiments of the present invention suggests that skin temperature may effect impedance and/or hydration measurements, and that skin temperature measurements may be used to correct impedance and/or hydration measurements. In many embodiments, increase in skin temperature or heat flux can be associated with increased vaso-dilation near the skin surface, such that measured impedance measurement decreased, even through the hydration of the patient in deeper tissues under the skin remains substantially unchanged. Thus, use of the temperature sensor can allow for correction of the hydration signals to more accurately assess the hydration, for example extra cellular hydration, of deeper tissues of the patient, for example deeper tissues in the thorax. In many embodiments, use of the temperature sensor may be used to monitor heat output of the ultrasonic transducers 113A-D, such that the electronics circuitry 130 will reduce or stop activation of the ultrasonic transducers 113A-D if temperature levels exceed a predetermined level.

Electronics circuitry 130 may comprise a processor 146. Processor 146 comprises a tangible medium, for example read only memory (ROM), electrically erasable programmable read only memory (EEPROM) and/or random access memory (RAM). Electronic circuitry 130 may comprise real time clock and frequency generator circuitry 148. In many embodiments, processor 136 may comprise the frequency generator and real time clock. The processor can be configured to control a collection and transmission of data from the impedance circuitry electrocardiogram circuitry and the accelerometer. In many embodiments, device 100 comprise a distributed processor system, for example with multiple processors on device 100.

In many embodiments, electronics components 130 comprise wireless communications circuitry 132 to communicate with remote center 106. Printed circuit board 120 may comprise an antenna to facilitate wireless communication. The antenna may be integral with printed circuit board 120 or may be separately coupled thereto. The wireless communication circuitry can be coupled to the impedance circuitry, the electrocardiogram circuitry and the accelerometer to transmit to a remote center with a communication protocol at least one of the fluid content level, hydration signal, the electrocardiogram signal or the inclination signal. In specific embodiments, wireless communication circuitry is configured to transmit the hydration signal, the electrocardiogram signal and the inclination signal to the remote center with a single wireless hop, for example from wireless communication circuitry 132 to intermediate device 102. The communication protocol comprises at least one of Bluetooth, Zigbee, WiFi, WiMax, IR, amplitude modulation or frequency modulation. In many embodiments, the communications protocol comprises a two way protocol such that the remote center is capable of issuing commands to control data collection.

Intermediate device 102 may comprise a data collection system to collect and store data from the wireless transmitter. The data collection system can be configured to communicate periodically with the remote center. The data collection system can transmit data in response to commands from remote center 106 and/or in response to commands from the adherent device.

Activity sensor 134 may comprise an accelerometer with at least one measurement axis, for example two or more measurement axes. In many embodiments, activity sensor 134 comprises three axis accelerometer 132A. Three axis accelerometer 132A may comprise an X-axis 134X, a Y-axis 134Y and a Z-axis 134Z with each axis sensitive to gravity such that the orientation of the accelerometer can be determined in relation to gravity. Three axis accelerometer 132A can be aligned with electrodes of adherent patch 110A. X-axis 134X can be aligned with X-axis 112X of adherent patch 110. Y-axis 134Y can be aligned with Y-axis 112Y of adherent patch 110. Z-axis 134Z can be aligned with Z-axis 112Z of adherent patch 110. Axes of accelerometer 132A can be aligned with axes of patch 110A, for example with connectors 122A-D, such that the axes of the accelerometer are aligned with adherent patch and/or the electrodes in a predetermined configuration. Although the axes of the patch and accelerometer are shown substantially parallel, the axes of the patch can be aligned with the axes of the accelerometer in a non-parallel configuration, for example an oblique configuration with oblique angles between axes of the accelerometer and axes of the adherent patch and/or electrodes.

Impedance circuitry 136 can generate both hydration data and respiration data. In many embodiments, impedance circuitry 136 is electrically connected to electrodes 112A-D in a four pole configuration, such that electrodes 112A and 112D comprise outer electrodes that are driven with a current and comprise force electrodes that force the current through the tissue. The current delivered between electrodes 112A and 112D generates a measurable voltage between electrodes 112B and 112C, such that electrodes 112B and 112C comprise inner, sense, electrodes that sense and/or measure the voltage in response to the current from the force electrodes. In many embodiments, electrodes 112B and 112C may comprise force electrodes and electrodes 112A and 112B may comprise sense electrodes. The voltage measured by the sense electrodes can be used to measure the impedance of the patient and determine the respiration rate and/or hydration of the patient.

FIG. 1D1 shows an equivalent circuit 152 that can be used to determine optimal frequencies for measuring patient hydration. Work in relation to embodiments of the present invention indicates that the frequency of the current and/or voltage at the force electrodes can be selected so as to provide impedance signals related to the extracellular and/or intracellular hydration of the patient tissue. Equivalent circuit 152 comprises an intracellular resistance 156, or R(ICW) in series with a capacitor 154, and an extracellular resistance 158, or R(ECW). Extracellular resistance 158 is in parallel with intracellular resistance 156 and capacitor 154 related to capacitance of cell membranes. In many embodiments, impedances can be measured and provide useful information over a wide range of frequencies, for example from about 0.5 kHz to about 200 KHz. Work in relation to embodiments of the present invention suggests that extracellular resistance 158 can be significantly related extracellular fluid and to cardiac decompensation, and that extracellular resistance 158 and extracellular fluid can be effectively measured with frequencies in a range from about 0.5 kHz to about 20 kHz, for example from about 1 kHz to about 10 kHz. In many embodiments, a single frequency can be used to determine the extracellular resistance and/or fluid. As sample frequencies increase from about 10 kHz to about 20 kHz, capacitance related to cell membranes decrease the impedance, such that the intracellular fluid contributes to the impedance and/or hydration measurements. Thus, many embodiments of the present invention measure hydration with frequencies from about 0.5 kHz to about 20 kHz to determine patient hydration.

In many embodiments, impedance circuitry 136 can be configured to determine respiration of the patient. In specific embodiments, the impedance circuitry can measure the hydration at 25 Hz intervals, for example at 25 Hz intervals using impedance measurements with a frequency from about 0.5 kHz to about 20 kHz.

ECG circuitry 138 can generate electrocardiogram signals and data from two or more of electrodes 112A-D in many ways. In many embodiments, ECG circuitry 138 is connected to inner electrodes 112B and 122C, which may comprise sense electrodes of the impedance circuitry as described above. In many embodiments, ECG circuitry 138 can be connected to electrodes 112A and 112D so as to increase spacing of the electrodes. The inner electrodes may be positioned near the outer electrodes to increase the voltage of the ECG signal measured by ECG circuitry 138. In many embodiments, the ECG circuitry may measure the ECG signal from electrodes 112A and 112D when current is not passed through electrodes 112A and 112D.

FIG. 1D2 shows adherent device 100 positioned on patient P to determine orientation of the adherent patch. X-axis 112X of device 100 is inclined at an angle α to horizontal axis Px of patient P. Z-axis 112Z of device 100 is inclined at angle α to vertical axis Pz of patient P. Y-axis 112Y may be inclined at a second angle, for example β, to anterior posterior axis Py and vertical axis Pz. As the accelerometer of adherent device 100 can be sensitive to gravity, inclination of the patch relative to axis of the patient can be measured, for example when the patient stands.

FIG. 1D3 shows vectors from a 3D accelerometer to determine orientation of the measurement axis of the patch adhered on the patient. A Z-axis vector 112ZV can be measured along vertical axis 112Z with an accelerometer signal from axis 134Z of accelerometer 132A. An X-axis vector 112XV can be measured along horizontal axis 112X with an accelerometer signal from axis 134X of accelerometer 132A. Inclination angle α can be determined in response to X-axis vector 112XV and Z-axis vector 112ZV, for example with vector addition of X-axis vector 112XV and Z-axis vector 112ZV. An inclination angle β for the patch along the Y and Z axes can be similarly obtained an accelerometer signal from axis 134Y of accelerometer 132A and vector 112ZV.

FIG. 1E shows batteries 150 positioned over the flex printed circuit board and electronic components as in FIG. 1D. Batteries 150 may comprise rechargeable batteries that can be removed and/or recharged. In many embodiments, batteries 150 can be removed from the adherent patch and recharged and/or replaced.

FIG. 1F shows a top view of a cover 162 over the batteries, electronic components and flex printed circuit board as in FIGS. 1A to 1E. In many embodiments, an electronics housing 160 may be disposed under cover 162 to protect the electronic components, and in many embodiments electronics housing 160 may comprise an encapsulant over the electronic components and PCB. In many embodiments, cover 162 can be adhered to adherent patch 110 with an adhesive 164 on an underside of cover 162. In many embodiments, electronics housing 160 may comprise a water proof material, for example a sealant adhesive such as epoxy or silicone coated over the electronics components and/or PCB. In many embodiments, electronics housing 160 may comprise metal and/or plastic. Metal or plastic may be potted with a material such as epoxy or silicone.

Cover 162 may comprise many known biocompatible cover, casing and/or housing materials, such as elastomers, for example silicone. The elastomer may be fenestrated to improve breathability. In many embodiments, cover 162 may comprise many known breathable materials, for example polyester, polyamide, nylon and/or elastane (Spandex™). The breathable fabric may be coated to make it water resistant, waterproof, and/or to aid in wicking moisture away from the patch.

FIG. 1G shows a side view of adherent device 100 as in FIGS. 1A to 1F. Adherent device 100 comprises a maximum dimension, for example a length 170 from about 4 to 10 inches (from about 100 mm to about 250 mm), for example from about 6 to 8 inches (from about 150 mm to about 200 mm). In many embodiments, length 170 may be no more than about 6 inches (no more than about 150 mm). Adherent device 100 comprises a thickness 172. Thickness 172 may comprise a maximum thickness along a profile of the device. Thickness 172 can be from about 0.2 inches to about 0.6 inches (from about 5 mm to about 15 mm), from about 0.2 inches to about 0.4 inches (from about 5 mm to about 10 mm), for example about 0.3 inches (about 7.5 mm).

FIG. 1H shown a bottom perspective view of adherent device 100 as in FIGS. 1A to 1G. Adherent device 100 comprises a width 174, for example a maximum width along a width profile of adherent device 100. Width 174 can be from about 2 to about 4 inches (from about 50 mm to 100 mm), for example about 3 inches (about 75 mm).

FIGS. 1I and 1J show a side cross-sectional view and an exploded view, respectively, of adherent device 100 as in FIGS. 1A to 1H. Device 100 comprises several layers. Gel 114A, or gel layer, is positioned on electrode 112A to provide electrical conductivity between the electrode and the skin. Electrode 112A may comprise an electrode layer. Gel 114A, or gel layer, is also positioned on ultrasonic transducer 113A to provide acoustic conductivity between the electrode and the skin. In many embodiments ultrasonic transducer 113A has a separate gel 114A from electrode 112A. In many embodiments ultrasonic transducer 113A does not include a gel 114A and is directly connected to the skin. In many embodiments ultrasonic transducer 113A and electrode 112A are not positioned adjacent to each other. Adherent patch 110 may comprise a layer of breathable tape 110T, for example a known breathable tape, such as tricot-knit polyester fabric. An adhesive 116A, for example a layer of acrylate pressure sensitive adhesive, can be disposed on underside 110A of adherent patch 110.

FIG. 1IA is close up view of View A of FIG. 1J. The ultrasonic transducer 113A may include a piezoelectric driver 113A1. The piezoelectric driver 113A1 may be constructed from a piezoelectric ceramic, polymer, or composite material. The piezoelectric driver 113A1 may be cylindrical in shape. The piezoelectric driver 113A1 may have a thickness which is equal to ½ a wavelength of a desired center frequency of the piezoelectric driver 113A1. The desired center frequency may range from 1-20 MHz. The ultrasonic transducer 113A may include a backing 113A2 to increase the depth of penetration of the ultrasonic transducer 113A. The backing material may have an acoustic impedance which is equal to the acoustic impendence of the piezoelectric driver 113A1. The ultrasonic transducer 113A may include a housing 113A3 which is cylindrical in shape. The housing 113A3 may comprise a polymer material. The face 113A4 piezoelectric driver 113A1 and an exterior of the housing 113A3 may be coated with a conductive material such that the face 113A4 is electrically coupled to the exterior of the housing 113A3. In many embodiments a separate conductor is coupled to the face 113A4 of the piezoelectric driver. In many embodiments the face 113A4 of the piezoelectric driver is preferentially shaped to focus the ultrasonic waves toward a target tissue and to disperse the ultrasonic waves away from the target tissue. The exterior of the housing 113A3 may further be electrically coupled to a first pole of connector 122E of the circuit board 120. The coated exterior of the housing 113A3 may be electrically isolated from a back 113A5 of the piezoelectric driver 113A1. The back 113A5 of the piezoelectric driver 113A1 may be electrically coupled to a second pole of connector 122E of the circuit board 120. The second pole of connector 122E may provide the back 113A5 of the piezoelectric driver 113A1 with electrical power. Gel 114A may also be acoustically impedance-matched to the center wavelength of the ultrasonic transducer 113A to keep signals exiting the gel 112A in phase with the signal exiting the ultrasonic transducer. The width of gel 114A may be a ¼ wavelength of the center frequency of the piezoelectric driver 113A1, or equally divisible by ¼ wavelengths. Gel 114A, may have an acoustic impedance value which is between an acoustic impedance value of a piezoelectric driver 113A1 and a human body, which may be 1.5×10⁶ kg/m².

FIGS. 1I1 and 1J1 show a side cross-sectional view and an exploded view, respectively, of embodiments of the adherent device with a temperature sensor affixed to the gel cover. In these embodiments, gel cover 180 extends over a wider area than in the embodiments shown in FIGS. 1I and 1J. Temperature sensor 177 is disposed over a peripheral portion of gel cover 180. Temperature sensor 177 can be affixed to gel cover 180 such that the temperature sensor can move when the gel cover stretches and tape stretch with the skin of the patient. Temperature sensor 177 may be coupled to temperature sensor circuitry 144 through a flex connection comprising at least one of wires, shielded wires, non-shielded wires, a flex circuit, or a flex PCB. This coupling of the temperature sensor allows the temperature near the skin to be measured though the breathable tape and the gel cover. The temperature sensor can be affixed to the breathable tape, for example through a cutout in the gel cover with the temperature sensor positioned away from the gel pads. A heat flux sensor can be positioned near the temperature sensor, for example to measure heat flux through to the gel cover, and the heat flux sensor coupled to heat flux circuitry similar to the temperature sensor.

In many embodiments adherent device comprises electrodes 112A1, 112B1, 112C1 and 112D1 configured to couple to tissue through apertures in the breathable tape 110T. Electrodes 112A1-D1 can be fabricated in many ways. For example, electrodes 112A1-D1 can be printed on a flexible connector 112F, such as silver ink on polyurethane. Ultrasonic transducers 113A1-D1 may be surface mounted to flexible connecter 112F. Breathable tape 110T comprise apertures (not shown). Electrodes 112A1-D1 and ultrasonic transducers 113A1-D1 are exposed to the gel through apertures of breathable tape 110T. Gel 114A-D can be positioned over electrodes 112A1-D1 and ultrasonic transducers 113A1-D1 and the respective portions of the breathable tape 110T apertures, so as to couple electrodes 112A1-D1 and ultrasonic transducers 113A1-D1 to the skin of the patient. In many embodiments ultrasonic transducers 113A1-D1 include separate gel layers. The flexible connector 112F comprising the electrodes can extend from under the gel cover to the printed circuit board to connect to the printed circuit boards and/or components supported thereon. For example, flexible connector 112F may comprise flexible connector 122A to provide strain relief, as described above.

In many embodiments, gel 114A, or gel layer, comprises a hydrogel that is positioned on electrode 112A to provide electrical conductivity between the electrode and the skin. In many embodiments, gel 114A comprises a hydrogel that provides a conductive interface between skin and electrode, so as to reduce impedance between electrode/skin interface. In many embodiments, gel may comprise water, glycerol, and electrolytes, pharmacological agents, such as beta blockers, ace inhibitors, diuretics, steroid for inflammation, antibiotic, antifungal agent. In specific embodiments the gel may comprise cortisone steroid. The gel layer may comprise many shapes, for example, square, circular, oblong, star shaped, many any polygon shapes. In specific embodiments, the gel layer may comprise at least one of a square or circular geometry with a dimension in a range from about 0.005″ to about 0.100″, for example within a range from about 0.015″-0.070″, in many embodiments within a range from about 0.015″-0.040″, and in specific embodiments within a range from about 0.020″-0.040″. In many embodiments, the gel layer of each electrode comprises an exposed surface area to contact the skin within a range from about 100 mm² to about 1500 mm², for example a range from about 250 mm² to about 750 mm², and in specific embodiments within a range from about 350 mm² to about 650 mm². Work in relation with embodiments of the present invention suggests that such dimensions and/or exposed surface areas can provide enough gel area for robust skin interface without excessive skin coverage. In many embodiments, the gel may comprise an adhesion to skin, as may be tested with a 1800 degree peel test on stainless steel, of at least about 3 oz/in, for example an adhesion within a range from about 5-10 oz/in. In many embodiments, a spacing between gels is at least about 5 mm, for example at least about 10 mm. Work in relation to embodiments of the present invention suggests that this spacing may inhibit the gels from running together so as to avoid crosstalk between the electrodes. In many embodiments, the gels comprise a water content within a range from about 20% to about 30%, a volume resistivity within a range from about 500 to 2000 ohm-cm, and a pH within a range from about 3 to about 5.

In many embodiments, the electrodes, for example electrodes 112A1-D1, may comprise an electrode layer. A 0.001″-0.005″ polyester strip with silver ink for traces can extend to silver/silver chloride electrode pads. In many embodiments, the electrodes can provide electrical conduction through hydrogel to skin, and in many embodiments may be coupled directly to the skin. Although at least 4 electrodes are shown, many embodiments comprise at least two electrodes, for example 2 electrodes. In many embodiments, the electrodes may comprise at least one of carbon-filled ABS plastic, silver, nickel, or electrically conductive acrylic tape. In specific embodiments, the electrodes may comprise at least one of carbon-filled ABS plastic, Ag/AgCl. The electrodes may comprise many geometric shapes to contact the skin, for example at least one of square, circular, oblong, star shaped, polygon shaped, or round. In specific embodiments, a dimension across a width of each electrodes is within a range from about 002″ to about 0.050″, for example from about 0.010 to about 0.040″. In many a surface area of the electrode toward the skin of the patient is within a range from about 25 mm² to about 1500 mm², for example from about 75 mm² to about 150 mm² In many embodiments, the electrode comprises a tape that may cover the gel near the skin of the patient. In specific embodiments, the two inside electrodes may comprise force, or current electrodes, with a center to center spacing within a range from about 20 to about 50 mm. In specific embodiments, the two outside electrodes may comprise measurement electrodes, for example voltage electrodes, and a center-center spacing between adjacent voltage and current electrodes is within a range from about 15 mm to about 35 mm. Therefore, in many embodiments, a spacing between inner electrodes may be greater than a spacing between an inner electrode and an outer electrode.

In many embodiments, adherent patch 110 may comprise a layer of breathable tape 110T, for example a known breathable tape, such as tricot-knit polyester fabric. In many embodiments, breathable tape 110T comprises a backing material, or backing 111, with an adhesive. In many embodiments, the patch adheres to the skin of the patient's body, and comprises a breathable material to allow moisture vapor and air to circulate to and from the skin of the patient through the tape. In many embodiments, the backing is conformable and/or flexible, such that the device and/or patch does not become detached with body movement. In many embodiments, backing can sufficiently regulate gel moisture in absence of gel cover. In many embodiments, adhesive patch may comprise from 1 to 2 pieces, for example 1 piece. In many embodiments, adherent patch 110 comprises pharmacological agents, such as at least one of beta blockers, ace inhibitors, diuretics, steroid for inflammation, antibiotic, or antifungal agent. In specific embodiments, patch 110 comprises cortisone steroid. Patch 110 may comprise many geometric shapes, for example at least one of oblong, oval, butterfly, dogbone, dumbbell, round, square with rounded corners, rectangular with rounded corners, or a polygon with rounded corners. In specific embodiments, a geometric shape of patch 110 comprises at least one of an oblong, an oval or round. In many embodiments, the geometric shape of the patch comprises a radius on each corner that is no less than about one half a width and/or diameter of tape. Work in relation to embodiments of the present invention suggests that rounding the corner can improve adherence of the patch to the skin for an extended period of time because sharp corners, for example right angle corners, can be easy to peel. In specific embodiments, a thickness of adherent patch 110 is within a range from about 0.001″ to about 0.020″, for example within a range from about 0.005″ to about 0.010″. Work in relation to embodiments of the present invention indicates that these ranges of patch thickness can improve adhesion of the device to the skin of the patient for extended periods as a thicker adhesive patch, for example tape, may peel more readily. In many embodiments, length 170 of the patch is within a range from about 2″ to about 10″, width 174 of the patch is within a range from about 1″ to about 5″. In specific embodiments, length 170 is within a range from about 4″ to about 8″ and width 174 is within a range from about 2″ to about 4″. In many embodiments, an adhesion to the skin, as measured with a 180 degree peel test on stainless steel, can be within a range from about 10 to about 100 oz/in width, for example within a range from about 30 to about 70 oz/in width. Work in relation to embodiments of the present invention suggests that adhesion within these ranges may improve the measurement capabilities of the patch because if the adhesion is too low, patch will not adhere to the skin of the patient for a sufficient period of time and if the adhesion is too high, the patch may cause skin irritation upon removal. In many embodiments adherent patch 110 comprises a moisture vapor transmission rate (MVTR, g/m²/24 hrs) per American Standard for Testing and Materials E-96 (ASTM E-96) is at least about 400, for example at least about 1000. Work in relation to embodiments of the present invention suggest that MVTR values as specified above can provide improved comfort, for example such that in many embodiments skin does not itch. In many embodiments, the breathable tape 110T of adherent patch 110 may comprise a porosity (sec./100 cc/in²) within a wide range of values, for example within a range from about 0 to about 200. The porosity of breathable tape 110T may be within a range from about 0 to about 5. The above amounts of porosity can minimize itching of the patient's skin when the patch is positioned on the skin of the patient. In many embodiments, the MVTR values above may correspond to a MVTR through both the gel cover and the breathable tape. The above MVTR values may also correspond to an MVTR through the breathable tape, the gel cover and the breathable cover. The MVTR can be selected to minimize patient discomfort, for example itching of the patient's skin.

In many embodiments, the breathable tape may contain and elute a pharmaceutical agent, such as an antibiotic, anti-inflammatory or antifungal agent, when the adherent device is placed on the patient.

In many embodiments, tape 110T of adherent patch 110 may comprise backing material, or backing 111, such as a fabric configured to provide properties of patch 110 as described above. In many embodiments backing 111 provides structure to breathable tape 110T, and many functional properties of breathable tape 110T as described above. In many embodiments, backing 111 comprises at least one of polyester, polyurethane, rayon, nylon, breathable plastic film; woven, nonwoven, spunlace, knit, film, or foam. In specific embodiments, backing 111 may comprise polyester tricot knit fabric. In many embodiments, backing 111 comprises a thickness within a range from about 0.0005″ to about 0.020″, for example within a range from about 0.005″ to about 0.010″.

In many embodiments, an adhesive 116A, for example breathable tape adhesive comprising a layer of acrylate pressure sensitive adhesive, can be disposed on underside 110A of patch 110. In many embodiments, adhesive 116A adheres adherent patch 110 comprising backing 111 to the skin of the patient, so as not to interfere with the functionality of breathable tape, for example water vapor transmission as described above. In many embodiments, adhesive 116A comprises at least one of acrylate, silicone, synthetic rubber, synthetic resin, hydrocolloid adhesive, pressure sensitive adhesive (PSA), or acrylate pressure sensitive adhesive. In many embodiments, adhesive 116A comprises a thickness from about 0.0005″ to about 0.005″, in specific embodiments no more than about 0.003″. Work in relation to embodiments of the present invention suggests that these thicknesses can allow the tape to breathe and/or transmit moisture, so as to provide patient comfort.

A gel cover 180, or gel cover layer, for example a polyurethane non-woven tape, can be positioned over patch 110 comprising the breathable tape. A PCB layer, for example flex printed circuit board 120, or flex PCB layer, can be positioned over gel cover 180 with electronic components 130 connected and/or mounted to flex printed circuit board 120, for example mounted on flex PCB so as to comprise an electronics layer disposed on the flex PCB layer. In many embodiments, the adherent device may comprise a segmented inner component, for example the PCB may be segmented to provide at least many flexibility. In many embodiments, the electronics layer may be encapsulated in electronics housing 160 which may comprise a waterproof material, for example silicone or epoxy. In many embodiments, the electrodes are connected to the PCB with a flex connection, for example trace 123A of flex printed circuit board 120, so as to provide strain relief between the electrodes 112A-D, and ultrasonic transducers 113A-D and the PCB.

Gel cover 180 can inhibit flow of gel 112A and liquid. In many embodiments, gel cover 180 can inhibit gel 112A from seeping through breathable tape 110T to maintain gel integrity over time. Gel cover 180 can also keep external moisture from penetrating into gel 112A. For example gel cover 180 can keep liquid water from penetrating though the gel cover into gel 112A, while allowing moisture vapor from the gel, for example moisture vapor from the skin, to transmit through the gel cover. The gel cover may comprise a porosity at least 200 sec./100 cc/in², and this porosity can ensure that there is a certain amount of protection from external moisture for the hydrogel.

In many embodiments, the gel cover can regulate moisture of the gel near the electrodes so as to keeps excessive moisture, for example from a patient shower, from penetrating gels near the electrodes. In many embodiments, the gel cover may avoid release of excessive moisture form the gel, for example toward the electronics and/or PCB modules. Gel cover 180 may comprise at least one of a polyurethane, polyethylene, polyolefin, rayon, PVC, silicone, non-woven material, foam, or a film. In many embodiments gel cover 180 may comprise an adhesive, for example a acrylate pressure sensitive adhesive, to adhere the gel cover to adherent patch 110. In specific embodiments gel cover 180 may comprise a polyurethane film with acrylate pressure sensitive adhesive. In many embodiments, a geometric shape of gel cover 180 comprises at least one of oblong, oval, butterfly, dogbone, dumbbell, round, square, rectangular with rounded corners, or polygonal with rounded corners. In specific embodiments, a geometric shape of gel cover 180 comprises at least one of oblong, oval, or round. In many embodiments, a thickness of gel cover is within a range from about 0.0005″ to about 0.020″, for example within a range from about 0.0005 to about 0.010″. In many embodiments, gel cover 180 can extend outward from about 0-20 mm from an edge of gels, for example from about 5-15 mm outward from an edge of the gels.

In many embodiments, the breathable tape of adherent patch 110 comprises a first mesh with a first porosity and gel cover 180 comprises a breathable tape with a second porosity, in which the second porosity is less than the first porosity to inhibit flow of the gel through the breathable tape.

In many embodiments, device 100 includes a printed circuitry, for example a printed circuitry board (PCB) module that includes at least one PCB with electronics component mounted thereon on and the battery, as described above. In many embodiments, the PCB module comprises two rigid PCB modules with associated components mounted therein, and the two rigid PCB modules are connected by flex circuit, for example a flex PCB. In specific embodiments, the PCB module comprises a known rigid FR4 type PCB and a flex PCB comprising known polyimide type PCB. In specific embodiments, the PCB module comprises a rigid PCB with flex interconnects to allow the device to flex with patient movement. The geometry of flex PCB module may comprise many shapes, for example at least one of oblong, oval, butterfly, dogbone, dumbbell, round, square, rectangular with rounded corners, or polygon with rounded corners. In specific embodiments the geometric shape of the flex PCB module comprises at least one of dogbone or dumbbell. The PCB module may comprise a PCB layer with flex PCB 120 can be positioned over gel cover 180 and electronic components 130 connected and/or mounted to flex PCB 120 so as to comprise an electronics layer disposed on the flex PCB. In many embodiments, the adherent device may comprise a segmented inner component, for example the PCB, for limited flexibility. The printed circuit may comprise polyester film with silver traces printed thereon.

In many embodiments, the electronics layer may be encapsulated in electronics housing 160. Electronics housing 160 may comprise an encapsulant, such as a dip coating, which may comprise a waterproof material, for example silicone and/or epoxy. In many embodiments, the PCB encapsulant protects the PCB and/or electronic components from moisture and/or mechanical forces. The encapsulant may comprise silicone, epoxy, other adhesives and/or sealants. In many embodiments, the electronics housing may comprising metal and/or plastic housing and potted with aforementioned sealants and/or adhesives.

In many embodiments, the electrodes are connected to the PCB with a flex connection, for example trace 123A of flex PCB 120, so as to provide strain relive between the electrodes 112A-D, ultrasonic transducers 113A-D and the PCB. In such embodiments, motion of the electrodes relative to the electronics modules, for example rigid PCB's 120A-D with the electronic components mounted thereon, does not compromise integrity of the electrode/hydrogel/skin contact. In many embodiments, the electrodes can be connected to the PCB and/or electronics module with a flex PCB 120, such that the electrodes and adherent patch can move independently from the PCB module. In many embodiments, the flex connection comprises at least one of wires, shielded wires, non-shielded wires, a flex circuit, or a flex PCB. In specific embodiments, the flex connection may comprise insulated, non-shielded wires with loops to allow independent motion of the PCB module relative to the electrodes.

In specific embodiments, cover 162 comprises at least one of polyester, 5-25% elastane/spandex, polyamide fabric; silicone, a polyester knit, a polyester knit without elastane, or a thermoplastic elastomer. In many embodiments cover 162 comprises at least 400% elongation. In specific embodiments, cover 162 comprises at least one of a polyester knit with 10-20% spandex or a woven polyamide with 10-20% spandex. In many embodiments, cover 162 comprises a water repellent coating and/or layer on outside, for example a hydrophobic coating, and a hydrophilic coating on inside to wick moisture from body. In many embodiments the water repellent coating on the outside comprises a stain resistant coating. Work in relation to embodiments of the present invention suggests that these coatings can be important to keep excessive moisture from the gels near the electrodes and to remove moisture from body so as to provide patient comfort.

In many embodiments, cover 162 can encase the flex PCB and/or electronics and can be adhered to at least one of the electronics, the flex PCB or adherent patch 110, so as to protect at least the electronics components and the PCB. Cover 162 can attach to adherent patch 110 with adhesive 116B. Cover 162 can comprise many known biocompatible cover materials, for example silicone. Cover 162 can comprise an outer polymer cover to provide smooth contour without limiting flexibility. In many embodiments, cover 162 may comprise a breathable fabric. Cover 162 may comprise many known breathable fabrics, for example breathable fabrics as described above. In many embodiments, the breathable cover may comprise a breathable water resistant cover. In many embodiments, the breathable fabric may comprise polyester, nylon, polyamide, and/or elastane (Spandex™) to allow the breathable fabric to stretch with body movement. In many embodiments, the breathable tape may contain and elute a pharmaceutical agent, such as an antibiotic, anti-inflammatory or antifungal agent, when the adherent device is placed on the patient.

The breathable cover 162 and adherent patch 110 comprise breathable tape can be configured to couple continuously for at least one week the at least one electrode to the skin so as to measure breathing of the patient. The breathable tape may comprise the stretchable breathable material with the adhesive and the breathable cover may comprises a stretchable breathable material connected to the breathable tape, as described above, such that both the adherent patch and cover can stretch with the skin of the patient. The breathable cover may also comprise a water resistant material. Arrows 182 show stretching of adherent patch 110, and the stretching of adherent patch can be at least two dimensional along the surface of the skin of the patient. As noted above, connectors 122A-H between PCB 130 and electrodes 112A-D, and ultrasonic transducers 113A-D may comprise insulated wires that provide strain relief between the PCB and the electrodes, such that the electrodes can move with the adherent patch as the adherent patch comprising breathable tape stretches. Arrows 184 show stretching of cover 162, and the stretching of the cover can be at least two dimensional along the surface of the skin of the patient.

Cover 162 can be attached to adherent patch 110 with adhesive 116B such that cover 162 stretches and/or retracts when adherent patch 110 stretches and/or retracts with the skin of the patient. For example, cover 162 and adherent patch 110 can stretch in two dimensions along length 170 and width 174 with the skin of the patient, and stretching along length 170 can increase spacing between electrodes. Stretching of the cover and adherent patch 110, for example in two dimensions, can extend the time the patch is adhered to the skin as the patch can move with the skin such that the patch remains adhered to the skin. Electronics housing 160 can be smooth and allow breathable cover 162 to slide over electronics housing 160, such that motion and/or stretching of cover 162 is slidably coupled with housing 160. The printed circuit board can be slidably coupled with adherent patch 110 that comprises breathable tape 110T, such that the breathable tape can stretch with the skin of the patient when the breathable tape is adhered to the skin of the patient, for example along two dimensions comprising length 170 and width 174.

The stretching of the adherent device 100 along length 170 and width 174 can be characterized with a composite modulus of elasticity determined by stretching of cover 162, adherent patch 110 comprising breathable tape 110T and gel cover 180. For the composite modulus of the composite fabric cover-breathable tape-gel cover structure that surrounds the electronics, the composite modulus may comprise no more than about 1 MPa, for example no more than about 0.3 MPa at strain of no more than about 5%. These values apply to any transverse direction against the skin.

The stretching of the adherent device 100 along length 170 and width 174, may also be described with a composite stretching elongation of cover 162, adherent patch 110 comprising breathable tape breathable tape 110T and gel cover 180. The composite stretching elongation may comprise a percentage of at least about 10% when 3 kg load is a applied, for example at least about 100% when the 3 kg load applied. These percentages apply to any transverse direction against the skin.

The printed circuit board may be adhered to the adherent patch 110 comprising breathable tape 110T at a central portion, for example a single central location, such that adherent patch 110 can stretch around this central region. The central portion can be sized such that the adherence of the printed circuit board to the breathable tape does not have a substantial effect of the modulus of the composite modulus for the fabric cover, breathable tape and gel cover, as described above. For example, the central portion adhered to the patch may be less than about 100 mm², for example with dimensions of approximately 10 mm by 10 mm (about 0.5″ by 0.5″). Such a central region may comprise no more than about 10% of the area of patch 110, such that patch 110 can stretch with the skin of the patient along length 170 and width 174 when the patch is adhered to the patient.

The cover material may comprise a material with a low recovery, which can minimize retraction of the breathable tape from the pulling by the cover. Suitable cover materials with a low recovery include at least one of polyester or nylon, for example polyester or nylon with a loose knit. The recovery of the cover material may be within a range from about 0% recovery to about 25% recovery. Recovery can refer to the percentage of retraction the cover material that occurs after the material has been stretched from a first length to a second length. For example, with 25% recovery, a cover that is stretched from a 4 inch length to a 5 inch length will retract by 25% to a final length of 4.75 inches.

Electronics components 130 can be affixed to printed circuit board 120, for example with solder, and the electronics housing can be affixed over the PCB and electronics components, for example with dip coating, such that electronics components 130, printed circuit board 120 and electronics housing 160 are coupled together. Electronics components 130, printed circuit board 120, and electronics housing 160 are disposed between the stretchable breathable material of adherent patch 110 and the stretchable breathable material of cover 160 so as to allow the adherent patch 110 and cover 160 to stretch together while electronics components 130, printed circuit board 120, and electronics housing 160 do not stretch substantially, if at all. This decoupling of electronics housing 160, printed circuit board 120 and electronic components 130 can allow the adherent patch 110 comprising breathable tape to move with the skin of the patient, such that the adherent patch can remain adhered to the skin for an extended time of at least one week, for example two or more weeks.

An air gap 169 may extend from adherent patch 110 to the electronics module and/or PCB, so as to provide patient comfort. Air gap 169 allows adherent patch 110 and breathable tape 110T to remain supple and move, for example bend, with the skin of the patient with minimal flexing and/or bending of printed circuit board 120 and electronic components 130, as indicated by arrows 186. Printed circuit board 120 and electronics components 130 that are separated from the breathable tape 110T with air gap 169 can allow the skin to release moisture as water vapor through the breathable tape, gel cover, and breathable cover. This release of moisture from the skin through the air gap can minimize, and even avoid, excess moisture, for example when the patient sweats and/or showers.

The breathable tape of adherent patch 110 may comprise a first mesh with a first porosity and gel cover 180 may comprise a breathable tape with a second porosity, in which the second porosity is less than the first porosity to minimize, and even inhibit, flow of the gel through the breathable tape. The gel cover may comprise a polyurethane film with the second porosity.

Cover 162 may comprise many shapes. In many embodiments, a geometry of cover 162 comprises at least one of oblong, oval, butterfly, dogbone, dumbbell, round, square, rectangular with rounded corners, or polygonal with rounded corners. In specific embodiments, the geometric of cover 162 comprises at least one of an oblong, an oval or a round shape.

Cover 162 may comprise many thicknesses and/or weights. In many embodiments, cover 162 comprises a fabric weight: within a range from about 100 to about 200 g/m², for example a fabric weight within a range from about 130 to about 170 g/m².

In many embodiments, cover 162 can attach the PCB module to adherent patch 110 with cover 162, so as to avoid interaction of adherent patch 110C with the PCB having the electronics mounted therein. Cover 162 can be attached to breathable tape 110T and/or electronics housing 160 comprising over the encapsulated PCB. In many embodiments, adhesive 116B attaches cover 162 to adherent patch 110. In many embodiments, cover 162 attaches to adherent patch 110 with adhesive 116B, and cover 162 is adhered to the PCB module with an adhesive 161 on the upper surface of the electronics housing. Thus, the PCB module can be suspended above the adherent patch via connection to cover 162, for example with a gap 169 between the PCB module and adherent patch. In many embodiments, gap 169 permits air and/or water vapor to flow between the adherent patch and cover, for example through adherent patch 110 and cover 162, so as to provide patient comfort.

In many embodiments, adhesive 116B is configured such that adherent patch 110 and cover 162 can be breathable from the skin to above cover 162 and so as to allow moisture vapor and air to travel from the skin to outside cover 162. In many embodiments, adhesive 116B is applied in a pattern on adherent patch 110 such that the patch and cover can be flexible so as to avoid detachment with body movement. Adhesive 116B can be applied to upper side 110B of patch 110 and comprise many shapes, for example a continuous ring, dots, dashes around the perimeter of adherent patch 110 and cover 162. Adhesive 116B may comprise at least one of acrylate, silicone, synthetic rubber, synthetic resin, pressure sensitive adhesive (PSA), or acrylate pressure sensitive adhesive. Adhesive 16B may comprise a thickness within a range from about 0.0005″ to about 0.005″, for example within a range from about 0.001-0.005″. In many embodiments, adhesive 116B comprises a width near the edge of patch 110 and/or cover 162 within a range from about 2 to about 15 mm, for example from about 3 to about 7 near the periphery. In many embodiments with such widths and/or thickness near the edge of the patch and/or cover, the tissue adhesion may be at least about 30 oz/in, for example at least about 40 oz/in, such that the cover remains attached to the adhesive patch when the patient moves.

In many embodiments, the cover is adhered to adherent patch 110 comprising breathable tape 110T at least about 1 mm away from an outer edge of adherent patch 110. This positioning protects the adherent patch comprising breathable tape 110T from peeling away from the skin and minimizes edge peeling, for example because the edge of the patch can be thinner. In many embodiments, the edge of the cover may be adhered at the edge of the adherent patch, such that the cover can be slightly thicker at the edge of the patch which may, in many instances, facilitate peeling of the breathable tape from the skin of the patient.

Gap 169 extend from adherent patch 110 to the electronics module and/or PCB a distance within a range from about 0.25 mm to about 4 mm, for example within a range from about 0.5 mm to about 2 mm.

In many embodiments, the adherent device comprises a patch component and at least one electronics module. The patch component may comprise adherent patch 110 comprising the breathable tape with adhesive coating 116A, at least one electrode, for example electrode 112A and gel 114. The at least one electronics module can be separable from the patch component. In many embodiments, the at least one electronics module comprises the flex printed circuit board 120, electronic components 130, electronics housing 160 and cover 162, such that the flex printed circuit board, electronic components, electronics housing and cover are reusable and/or removable for recharging and data transfer, for example as described above. In many embodiments, adhesive 116B is coated on upper side 110A of adherent patch 110B, such that the electronics module can be adhered to and/or separated from the adhesive component. In specific embodiments, the electronic module can be adhered to the patch component with a releasable connection, for example with Velcro™, a known hook and loop connection, and/or snap directly to the electrodes. Two electronics modules can be provided, such that one electronics module can be worn by the patient while the other is charged, as described above. Many patch components can be provided for monitoring over the extended period. For example, about 12 patches can be used to monitor the patient for at least 90 days with at least one electronics module, for example with two reusable electronics modules.

In many embodiments, the adherent device comprises a patch component and at least one electronics module. The patch component may comprise adherent patch 110 comprising the breathable tape with adhesive coating 116A, at least one electrode, for example electrode 112A and gel 114. The at least one electronics module can be separable from the patch component. In many embodiments, the at least one electronics module comprises the flex printed circuit board 120, electronic components 130, electronics housing 160 and cover 162, such that the flex printed circuit board, electronic components, electronics housing and cover are reusable and/or removable for recharging and data transfer, for example as described above. In many embodiments, adhesive 116B is coated on upper side 110A of adherent patch 110B, such that the electronics module can be adhered to and/or separated from the adhesive component. In specific embodiments, the electronic module can be adhered to the patch component with a releasable connection, for example with Velcro™, a known hook and loop connection, and/or snap directly to the electrodes. Two electronics modules can be provided, such that one electronics module can be worn by the patient while the other is charged, as described above. Many patch components can be provided for monitoring over the extended period. For example, about 12 patches can be used to monitor the patient for at least 90 days with at least one electronics module, for example with two reusable electronics modules.

At least one electrode 112A can extend through at least one aperture 180A in the breathable tape 110 and gel cover 180.

In many embodiments, the adhesive patch may comprise a medicated patch that releases a medicament, such as antibiotic, beta-blocker, ACE inhibitor, diuretic, or steroid to reduce skin irritation. The adhesive patch may comprise a thin, flexible, breathable patch with a polymer grid for stiffening. This grid may be anisotropic, may use electronic components to act as a stiffener, may use electronics-enhanced adhesive elution, and may use an alternating elution of adhesive and steroid.

FIG. 1K shows at least one electrode 190 configured to electrically couple to a skin of the patient through a breathable tape 192. In many embodiments, at least one electrode 190 and breathable tape 192 comprise electrodes and materials similar to those described above. Electrode 190 and breathable tape 192 can be incorporated into adherent devices as described above, so as to provide electrical coupling between the skin an electrode through the breathable tape, for example with the gel.

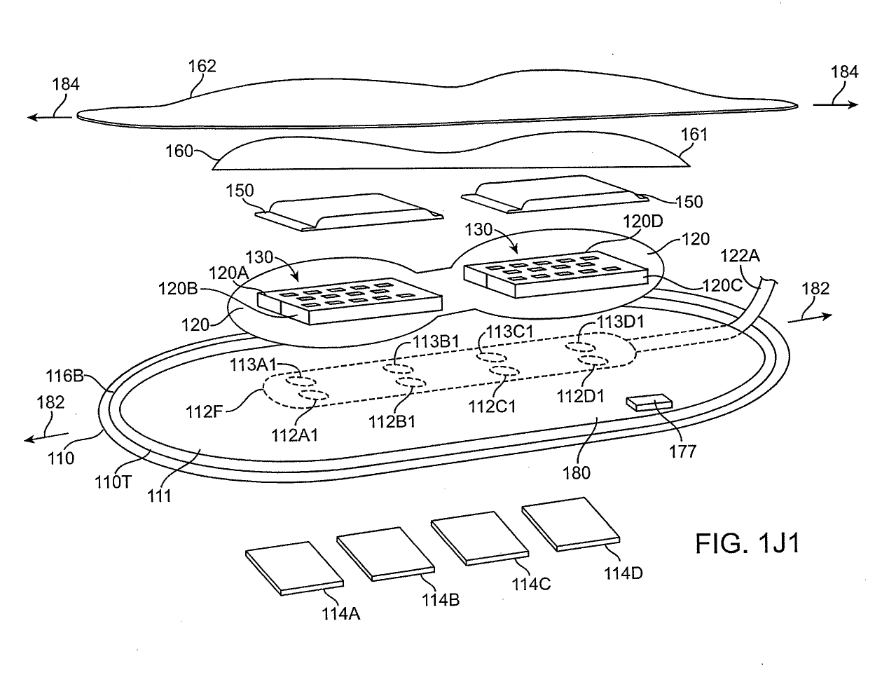

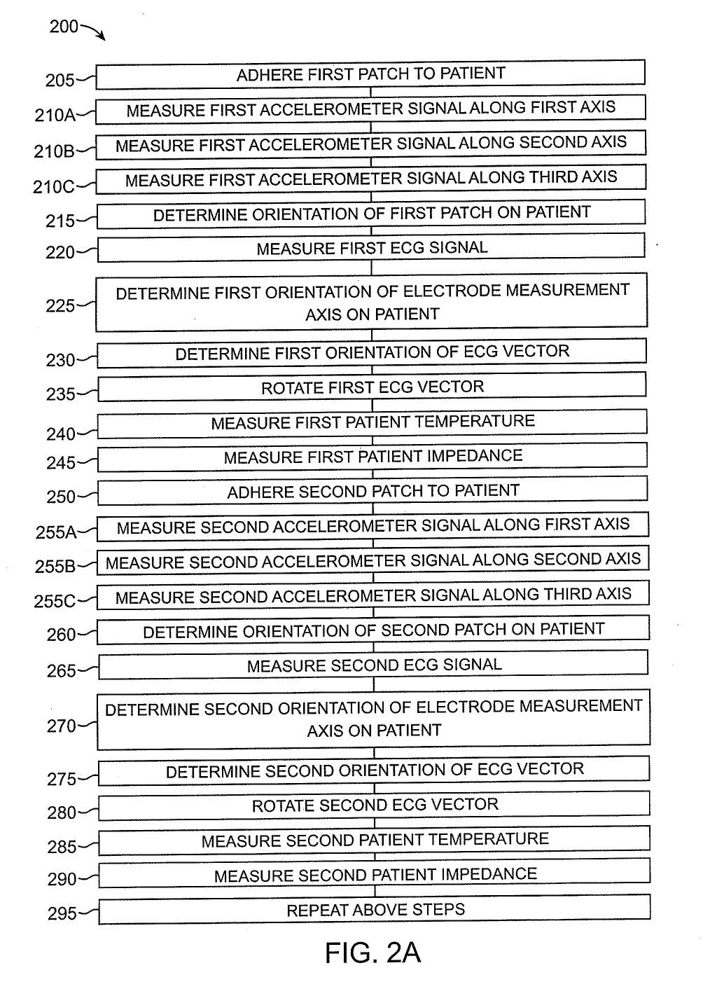

FIG. 2A shows a method 200 of monitoring a patient. A step 205 adheres a first adherent patch to the patient, for example an adherent patch as described above. The first adherent patch may comprise a first patch that is separable from an electronics module, as described above. The first adherent patch may comprise a first patch of a first device with the electronics module fixed to the adherent patch, for example disposable electronics with a disposable patch.

A step 210A measures a first accelerometer signal along a first axis, for example an X-axis of a 3D accelerometer responsive to gravity as described above. A step 210B measures a first accelerometer signal along a second axis, for example a y-axis of a 3D accelerometer as described above. A step 210C measures a first accelerometer signal along a third axis, for example a Z-axis of a 3D accelerometer as described above. Measurement of the accelerometer signal with step 210A, step 210B and step 21C, which may comprise sub-steps, can be performed with the patient in a known and/or determined position. The patient may be asked to stand and/or sit upright in a chair and the first signal measured. In many embodiments, the 3D accelerometer signal can be analyzed to determine that the patient is standing, walking and the first signal determined from a plurality of measurements to indicate that the patient is upright for the measurement of the first signal.

A step 215 determines an orientation of the first patch on the patient. The accelerometer can be coupled to the patch with a pre-determined orientation, for example with connectors as described above, such that the orientation of the patch can be determined from the accelerometer signal and the orientation of the 3D accelerometer on the adherent patch and the orientation of the patient.

A step 220 measures a first ECG signal. The first ECG signal can be measured with the electrodes attached to the patient when the patch comprises the first orientation. The ECG signal can be measured with electronics components and electrodes, as described above.

A step 225 determines a first orientation of an electrode measurement axis on the patient. The electrode measurement axis may correspond to one of the measurement axes of the 3D accelerometer, for example an X-axis of the accelerometer as described above. However, the orientation of the electrode measurement axis can be aligned in relation to the axes of the accelerometer in many ways, for example at oblique angles, such that the alignment of the accelerometer with the electrode measurement axis is known and the signal from the accelerometer can be used to determine the alignment of the electrode measurement axis.

A step 230 determines a first orientation of the ECG vector. The orientation of the ECG vector can be determined in response to the polarity of the measurement electrodes and orientation of the electrode measurement axis, as described above.

A step 235 rotates a first ECG vector. The first ECG vector orientation of the ECG vector can be used to rotate the ECG vector onto a desired axis, for example an X-axis of the patient in response to the first orientation of the ECG vector and the accelerometer signal. For example, if the first measurement axis of the first ECG vector is rotated five degrees based on the accelerometer signal, the first ECG vector can be rotated by five degrees so as to align the first ECG vector with the patient axis.

A step 240 measures a first patient temperature. The first temperature of the patient can be measured with electronics of the adherent device, as described above.

A step 245 measures a first patient impedance. The first patient impedance may comprise a four pole impedance measurement, as described above. The first patient impedance can be used to determine respiration of the patient and/or hydration of the patient.

A step 250 adheres a second patch to the patient. The second patch may comprise a second patch connected to a reusable electronics module, for example a reusable electronics module connected to the first patch for the first patient measurements above. The second patch may comprise a second patch of a second adherent device comprising a second electronics module in which the second patch and second electronics module comprise a disposable second adherent device and the first adherent patch and first electronics module comprise a first disposable adherent device.

A step 255A measures a second accelerometer signal along a first axis, for example an x-axis of the accelerometer as described above. The first axis may comprise the first axis of the first accelerometer as described above, for example the X-axis of the accelerometer used to measure the X-axis signal with the first measurement. In many embodiments, the second accelerometer signal along the first axis may comprise an X-axis of a second accelerometer, for example a second disposable electronics module, aligned with an electrode measurement axis as described above.

A step 255B measures a second accelerometer signal along a second axis. The second axis may comprise the second axis of the first accelerometer as described above, for example the Y-axis of the accelerometer used to measure the Y-axis signal with the first measurement. In many embodiments, the second accelerometer signal along the second axis may comprise a Y-axis of a second accelerometer, for example a second disposable electronics module, aligned with an electrode measurement axis as described above.

A step 255C measures a second accelerometer signal along a third axis. The third axis may comprise the third axis of the first accelerometer as described above, for example the Z-axis of the accelerometer used to measure the Z-axis signal with the first measurement. In many embodiments, the second accelerometer signal along the third axis may comprise a Z-axis of a second accelerometer, for example a second disposable electronics module, aligned with an electrode measurement axis as described above.

A step 260 determines an orientation of the second patch on the patient. The accelerometer can be coupled to the second patch with a pre-determined orientation, for example with connectors as described above, such that the orientation of the second patch can be determined from the second accelerometer signal and the orientation of the 3D accelerometer on the adherent patch and the orientation of the patient.

A step 265 measures a second ECG signal. The second ECG signal can be measured with the electrodes attached to the patient when the second patch comprises the second orientation, for example after the first patch has been removed and the second patch has been positioned on the patient as described above. The ECG signal can be measured with electronics components and electrodes, as described above.

A step 270 determines a second orientation of the electrode measurement axis on the patient. The second orientation of the electrode measurement axis may comprise orientation of an axis of a second set of electrodes, for example a second set of electrodes disposed along an axis of the second patch. The second orientation of the electrode measurement axis may correspond to one of the measurement axes of the 3D accelerometer, for example an X-axis of the accelerometer as described above. However, the second orientation of the electrode measurement axis can be aligned in relation to the axes of the accelerometer in many ways, for example at oblique angles, such that the alignment of the accelerometer with the second electrode measurement axis is known and the signal from the accelerometer can be used to determine the alignment of the electrode measurement axis.

A step 275 determines a second orientation of the ECG vector. The second orientation of the ECG vector can be determined in response to the polarity of the second measurement electrodes and second orientation of the electrode measurement axis, for example second measurement electrodes on the second adherent patch that extend along the electrode measurement axis of the second adherent patch.

A step 280 rotates a second ECG vector. The second ECG vector orientation of the second ECG vector can be used to rotate the second ECG vector onto the desired axis, for example the X-axis of the patient in response to the first orientation of the ECG vector and the accelerometer signal. For example, if the first measurement axis of the first ECG vector is rotated five degrees from the X-axis based on the accelerometer signal, the first ECG vector can be rotated by five degrees so as to align the first ECG vector with the X-axis of the patient, for example the horizontal axis of the patient.

A step 285 measures a second patient temperature. The second temperature of the patient can be measured with electronics of the adherent device, as described above.

A step 290 measures a second patient impedance. The second patient impedance may comprise a four pole impedance measurement, as described above. The second patient impedance can be used to determine respiration of the patient and/or hydration of the patient.

A step 295 repeats the above steps. The above steps can be repeated to provide longitudinal monitoring of the patient with differential measurement of patient status. The monitoring of the patient may comprise a comparison of baseline patient data with subsequent patient date.

Many of the steps of method 200 can be performed with the processor system, as described above.

It should be appreciated that the specific steps illustrated in FIG. 2A provides a particular method of monitoring a patient, according to an embodiment of the present invention. Other sequences of steps may also be performed according to alternative embodiments. For example, alternative embodiments of the present invention may perform the steps outlined above in a different order. Moreover, the individual steps illustrated in FIG. 2A may include multiple sub-steps that may be performed in various sequences as appropriate to the individual step. Furthermore, additional steps may be added or removed depending on the particular applications. One of ordinary skill in the art would recognize many variations, modifications, and alternatives.

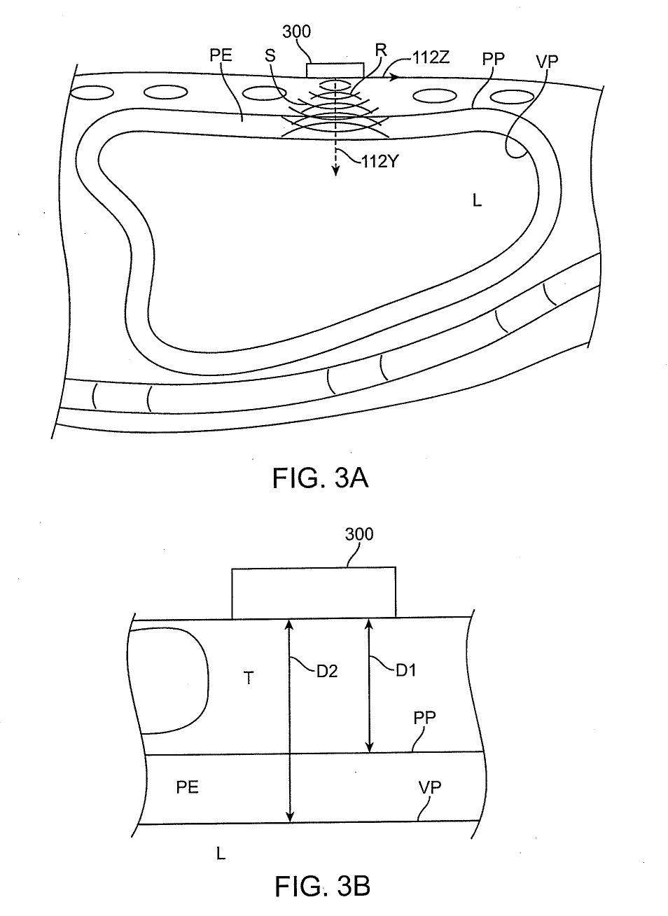

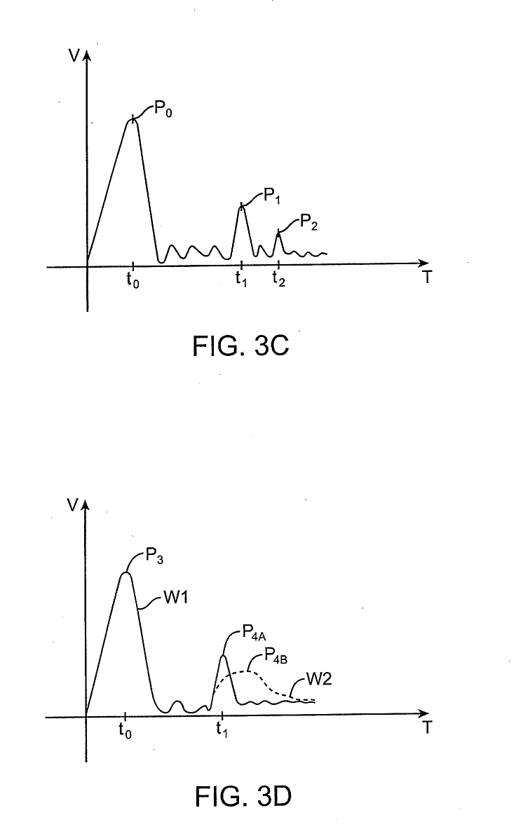

FIG. 3A shows a cross-sectional view of an adherent device 300 for detecting fluid content in a portion of a body of a patient. The adherent device 300 may include at least one ultrasound transducer and share the construction of the adherent devices disclosed herein. The adherent device 300 is shown adhered to the skin of a patient about a torso region. The adherent device 300 may transmit an ultrasonic signal S into the body of the patient, and receive back a portion of reflected signals R. The reflected signals R may be reflected from the visceral pleura VP and parietal pleura PP of the lung L. The adherent device may be configured to calculate the distance from the visceral pleura VP and/or the parietal pleura PP, to the adherent device 300. The adherent device may also be configured to determine the presence and quantify a pleural edema by measuring a fluid buildup in the lung tissue behind visceral pleura.

FIG. 3B shows a close-up portion of FIG. 3A. Distance D1 represents the distance from the adherent device 300 to the parietal pleura PP. Distance D2 represents the distance from the adherent device 300 to the visceral pleura VP. Distance D1 may generally stay constant during a pleural effusion PE, as tissue T is much less compressible than the lung L. Accordingly, a pressure increase from a pleural effusion PE may cause distance D2 to noticeably increase. Distances D1 and D2 may be calculated by transmitting an ultrasonic wave and receiving reflected waves from the parietal pleura PP and visceral pleura VP, respectively, at different time intervals t₁ and t₂ respectively, using the speed of sound in the tissue and the pleural effusion PE. The speed of sound in the tissue and pleural effusion PE fluid are known or measurable quantities. The speed of sound of tissue eₜ may be between 1450 and 1600 m/s for some tissues, and the speed of sound for pleural effusion fluid eₚₑ may be approximately 1400 m/s. The speed of sound in the tissue T and PE may be experimentally determined as well. Accordingly, D1 may be calculated by the simplified equation: D1=eₜ/t₁. D2 may be calculated by the simplified equation: D2=D1+(t₂−t₁)/eₚₑ. This example is simplified and does not take into account other tissues, for example, e.g., fat and bone, which a commonly skilled artisan would be aware of and account for.

FIG. 3C shows a graphical output that may be obtained with an experiment for detecting fluid content in a portion of a body of a patient. The patient may having pulmonary with detectable pleural effusion, in which the ultrasound transducer is positioned in a location sensitive to accumulation of liquid in the pleura. A graph is shown, of ultrasonic signals received by the adherent device 300, and plotted with respect to voltage V and time T. Ultrasonic waves can be reflected when there is an impedance change, for example with a change in material. Peaks P₀, P₁, and P₂ represent voltage peaks generated by the piezoelectric ceramic material from reflected waves. Peak P₀ may represent the reflection off the adherent device/skin interface of the patient. Peak P₁ and P₂ may represent reflections off surfaces inside a human body. Peak P₁ may represent a reflected wave from the parietal pleura PP, and peak P₂ may represent a reflected wave from the visceral pleura VP. The distances of the parietal pleura PP and visceral pleura VP may be calculated as described above. A gap between peak P₁ and peak P₂ may represent a pleural effusion PE.

Peaks P₁ and P₂ may be used to derive or calculate a fluid content signal. In many embodiments the adherent device 300 may be configured to discern the parietal pleura PP from the visceral pleura VP to determine a pleural effusion is present. The normal distance between the parietal pleura and visceral pleura is usually non-discernable, as a gap with only a thickness of 10-20 μm exists there-between. Thus, a measurement on a patient without a pleural effusion may only show pulse P₁. Work in relation to embodiments of the present invention suggests that pleural effusion may result in a separation of parietal pleura PP from visceral pleura VP, such that an ultrasound signal from the visceral pleura is reflected at an increased tissue penetration depth of the ultrasound signal, such that the presence of pulmonary edema can be determined in response to the tissue penetration depth of the ultrasound signal. Accordingly, the distance between the visceral pleura VP and the parietal pleura PP may not need be calculated, but only discerned, as the mere detection of the visceral pleura VP may indicate a pleural effusion. In many embodiments the adherent device is configured to routinely scan an area of a patient's body to detect a pleural effusion by detecting the presence of the visceral pleura VP, and to determine the rate of a pleural effusion volume increase. In many embodiments the adherent device is configured to save data regarding routine scans in memory, for comparison with later routine scans, to determine the buildup of pleural fluid in the lungs. Accordingly, a pleural effusion may be detected before the pleural effusion becomes clinically noticeable by the patient.

In many embodiments the adherent device 300 is configured to scan an area of an area of a patient's body to detect a pleural effusion in conjunction with other sensor measurements. In many embodiments the adherent device 300 the adherent device 300 is configured to detect a pleural effusion when the patient's body is in a predetermined position. The predetermined position may be determined by the accelerometer, and method for using, as disclosed herein. A pleural effusion often will move within the pleural space under the influence of gravity, and thus may be difficult to detect unless a very large amount of fluid, is present. Pleural effusion volumes of 75-350 ml can result in a deformation of the costophrenic angle of the lung above the diaphragm, as liquid has a tendency to gather in that area in certain bodily positions such as sitting and standing. In many embodiments the adherent device 300 may be coupled to the skin of the patient outside the region of the costophrenic angle of the lung and configured the detect pleural effusions when a predetermined body position is sensed, for example, when the patient is upright. Pleural fluid may also gather in certain positions during a patient's slumber, for example, fluid may accumulate in the right most position of the right lung when the patient sleeps on their right side. In many embodiments the adherent device 300 may be coupled to the skin of the patient about the bottommost region of the lung with respect to the patient's preferred sleeping position and configured the detect pleural effusions when a predetermined body position is sensed, and/or when a state of slumber is sensed. In many embodiments the adherent device 300 may be configured to detect pleural effusions when the lungs are in a maximum exhale or inhale position, which may be determined by separate sensor. In many embodiments a fluid content signal may be extrapolated to determine the volume of the pleural effusion. In many embodiments the adherent device 300 may coordinated to detect pleural effusions when other sensors of the adherent device indicate cardiac decompensation, for example, ECG, tissue resistance, bioimpedance, respiration, respiration rate variability, heart rate, heart rhythm, heart rate variability, heart rate turbulence, heart sounds, respiratory sounds, blood pressure, activity, posture, wake, sleep, orthopnea, temperature, heat flux, and weight sensors.

FIG. 3D shows a graphical output that may be obtained with an experiment for determining an amount fluid present in a portion of a body of a patient, such as the lung of the patient. A graph is shown, similar to as shown in FIG. 3C, of ultrasonic signals received by the adherent device 300, and plotted with respect to voltage V and time T. In cases of a pulmonary edema fluid may build up in the lung tissue comprising alveoli, which may also be accompanied by a pleural effusion. For example, there may be localized areas of plural effusions which are located away from the adherent device. For example, the alveoli of the lungs may comprise more liquid than normal and may even be filled with liquid. The liquid of the alveoli is less likely to shift compared to the liquid of a pleural effusion, as the liquid of the alveoli may be confined to the small sacs of the alveoli. Thus, there may be areas in the lung which show a pulmonary edema due to increased fluid of the alveoli and no pleural effusion. Pulses P₃, P_{4A}, and P_{4B} represents voltage peaks generated by the piezoelectric ceramic material from reflected waves. Pulses P₃ and P_{4A} represent reflections off surfaces inside a human body. Two wave forms are shown imposed over each other. The first waveform W1 (solid) represents a signal return from a normal patient. As shown, P₃ may represent the reflection off the adherent device/skin interface of the patient, and P_{4A} may represent a reflected wave from the lung tissue comprising the parietal pleura PP, visceral pleura VP, alveoli adjacent the pleura, or a combination thereof. In a lung which does not have a pulmonary edema, the lung tissue will offer poor signal reflectivity past the near the pleura, as the alveoli of the lung tissue comprise many pockets of air. The air pockets of the outer alveoli may substantially reflect and disperse the ultrasound signal such that penetration of the ultrasound waveform into the healthy lung is substantially degraded, and thus tissue away from the pleura will not effectively reflect an ultrasonic waveform. Accordingly, the waveform degrades rapidly past pulse P_{4A} of the first waveform W1, for a healthy lung. The second waveform W2 (dashed) represents a signal return from a patient suffering from a pulmonary edema. The lung tissue in the case of a pulmonary edema may be able to provide a reflective signal at increased tissue penetration depths, as portions of the lung tissue which are filled with liquid can allow increased transmission of the ultrasound wave so as to reflect the ultrasonic signal from an increased tissue penetration depth. The dotted portion of waveform W2 represents a portion of tissue with increased liquid in the alveoli, for example filled with liquid, as a result of a pulmonary edema. As shown, P_{4B} may represent a reflection off the lung tissue at an increased depth due to increase penetration of the ultrasound waveform due to decreased dispersion of the waveform so as to increase reflection at greater tissue depths. Pulse P_{4B} may comprise a lower amplitude and broader shape than P_{4A}, due to reduced acoustic impedance mismatch. The tissue penetration depth of the ultrasound signal and resulting reflection can increase with increased hydration of the alveoli. In a worst case situation, the entire lung may be filled with liquid and provide a much broader reflective signal at a much greater depth than shown in pulse P_{4B}. In use, the adherent device can be used to characterize the lung tissue over time so as to indicate a pulmonary edema by detecting a change of the profile of pulse P_{4A} into a profile which may be similar to pulse P_{4B}, and also output the characterization as a fluid content signal. A person or ordinary skill in the art can conduct empirical studies on an empirical number of patients, so as to characterize the return signal and correlate the return signal to determine the amount of hydration of the lungs, for example water content of the alveoli.

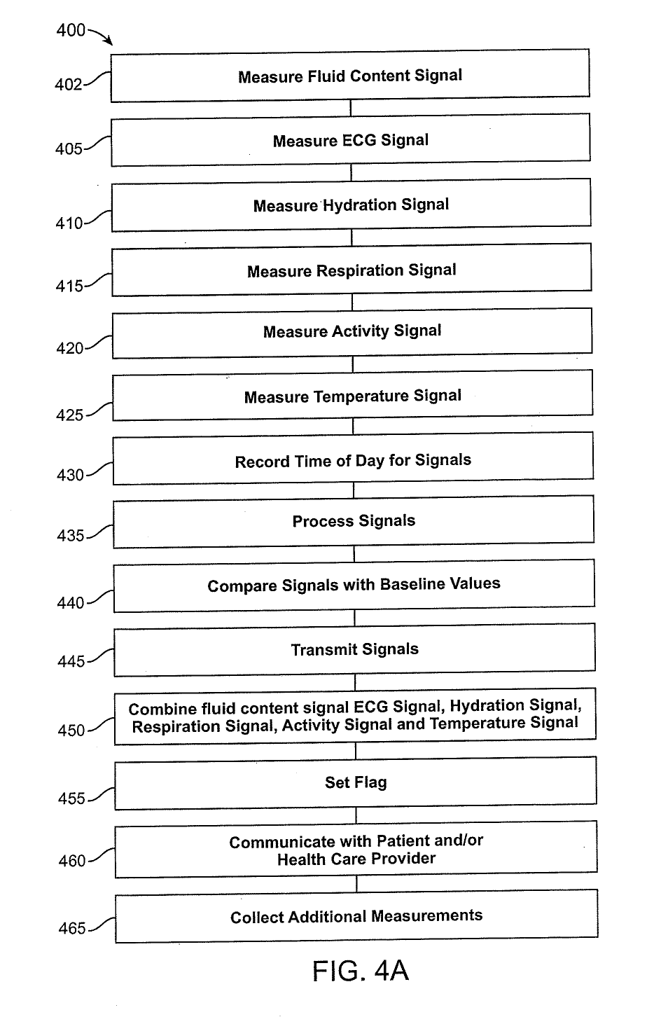

FIG. 4A shows a method 400 of detecting a pleural effusion for predicting an impending or risk of cardiac decompensation. A step 402 detects a fluid content signal regarding a pleural effusion as described herein. In many embodiments the fluid content signal is only measured when other sensors, such as activity sensors, determine a predetermined body position and/or breathing cycle and/or slumber status. A step 405 optionally measures an ECG signal. The ECG signal may comprise a differential signal measured with at least two electrodes and may be measured in many known ways. A step 410 optionally measures an hydration signal. The hydration signal may comprise an impedance signal, for example a four pole impedance signal, and may be measured in many known ways. A step 415 optionally measures a respiration signal. The respiration signal may comprise an impedance signal, and may be measured in many known ways. A step 420 optionally measures an activity signal. The activity signal may be measured in many known ways and may comprise a three dimensional accelerometer signal to determine a position of the patient, for example from a three dimensional accelerometer signal as described in FIG. 2A. A step 425 optionally measures a temperature signal. The temperature signal may be measured in many ways, for example with a thermistor, a thermocouple, and known temperature measurement devices. A step 430 optionally records a time of day of the signals, for example a local time of day such as morning, afternoon, evening, and/or nighttime.

A step 435 processes the signals. The signals may be processed in many known ways, for example to generate at least one of a derived signal, a time averaged signal, a filtered signal. In some embodiments, the signals may comprise raw signals. The fluid content signal may comprise a voltage signal generated by a piezoelectric transducer. The ECG signal may comprise at least one of a heart rate signal, a heart rate variability signal, an average heart rate signal, a maximum heart rate signal or a minimum heart rate signal. The hydration signal may comprise an impedance measurement signal. The activity signal may comprise at least one of an accelerometer signal, a position signal indicating the orientation of the patient, such as standing, lying, or sitting. The respiration signal may comprise a least one of a respiration rate, a maximum respiration rate, a minimum respiration rate, an average respiration rate or respiration rate variability. The temperature may comprise an average temperature or a peak temperature.

A step 440 compares the signals with baseline values. In many embodiments, the baseline values may comprise measurements from the same patient at an earlier time. In some embodiments, the baseline values comprise values for a patient population. In some embodiments, the baseline values for a patient population may comprise empirical data from a suitable patient population size, for example at least about 144 patients, depending on the number of variables measured, statistical confidence and power used. The measured signals may comprise changes and/or deviations from the baseline values.

A step 445 transmits the signals. In many embodiments, the measurement signals, which may comprise derived and/or processed measurement signals, are transmitted to the remote site for comparison. In some embodiments, the signals may be transmitted to a processor supported with the patient for comparison.

A step 450 optionally combines at least two of the fluid content signal, the ECG signal, the hydration signal, the respiration signal, the activity signal and the temperature signal to detect the impending decompensation. In many embodiments, at least three of the signals are combined. In some embodiments, at least four signals comprising the fluid content signal, the ECG signal, the respiration signal and the activity signal are combined to detect the impending decompensation. In specific embodiments, at least five signals comprising the fluid content signal, the ECG signal, the hydration signal, the activity signal and the temperature signal are combined to detect the impending decompensation.

The signals can be combined in many ways. In some embodiments, the signals can be used simultaneously to determine the impending cardiac decompensation.

In some embodiments, the signals can be combined by using the at least two of the electrocardiogram signal, the hydration signal, the respiration signal or the activity signal to look up a value in a previously existing array.

TABLE 1
Lookup Table for the Fluid Content Signals

| | Fluid Content Signal | | |
|---|---|---|---|
| Incidence of Pleural Effusions | A | B | C |
| Low | N | N | Y |
| Medium | N | Y | Y |
| High | Y | Y | Y |

Table 1 shows combination of the incidence of pleural effusions with fluid content signals to look up a value in a pre-existing array. For example, at a fluid content signal of A and an incidence of arrhythmias of “High,” the value in the table may comprise Y. In specific embodiments, the values of the look up table can be determined in response to empirical data measured for a patient population of at least about 100 patients, for example measurements on about 1000 to 10,000 patients. The incidence of pleural effusions can be determined in many ways, for example based on the number of pleural effusions over time, for example number per year or life. The incidence of pleural effusions can also be determined with an index that is determined in response to the volume and/or severity of the pleural effusions, for example with calculations that include the volume of the pleural effusions and/or severity of the pleural effusions. A pleural effusion in relation to embodiments of the present invention suggest that the presence of a pleural effusion can indicate the risk of an impending decompensation.

In some embodiments, the table may comprise a three or more dimensional look up table.

In some embodiments, the signals may be combined with at least one of adding, subtracting, multiplying, scaling or dividing the at least two of the electrocardiogram signal, the hydration signal, the respiration signal or the activity signal. In specific embodiments, the measurement signals can be combined with positive and or negative coefficients determined in response to empirical data measured for a patient population of at least about 100 patients, for example data on about 1000 to 10,000 patients.

In some embodiments, a weighted combination may combine at least 3 measurement signals to generate an output value according to a formula of the general form

OUTPUT=aX+bY+cZ

where a, b, and c comprise positive or negative coefficients determined from empirical data and X, Y and Z comprise measured signals for the patient, for example at least three of the electrocardiogram signal, the fluid content signal, the respiration signal or the activity signal. While three coefficients and three variables are shown, the data may be combined with multiplication and/or division. One or more of the variables may be the inverse of a measured variable.

In some embodiments, the ECG signal comprises a heart rate signal that can be divided by the activity signal. Work in relation to embodiments of the present invention suggest that an increase in heart rate with a decrease in activity can indicate an impending decompensation. The signals can be combined to generate an output value with an equation of the general form

OUTPUT=aX/Y+bZ

where X comprise a heart rate signal, Y comprises a hydration rate signal and Z comprises a fluid content signal, with each of the coefficients determined in response to empirical data as described above.

In some embodiments, the data may be combined with a tiered combination. While many tiered combinations can be used a tiered combination with three measurement signals can be expressed as

OUTPUT=(ΔX)+(ΔY)+(ΔZ)

where (ΔX), (ΔY), (ΔZ) may comprise change in heart rate signal from baseline, change in fluid content signal from baseline and change in respiration signal from baseline, and each may have a value of zero or one, based on the values of the signals. For example if the heart rate increase by 10%, (ΔX) can be assigned a value of 1. If fluid content increases by 5%, (ΔY) can be assigned a value of 1. If activity decreases below 10% of a baseline value (ΔZ) can be assigned a value of 1. When the output signal is three, a flag may be set to trigger an alarm.

In some embodiments, the data may be combined with a logic gated combination. While many logic gated combinations can be used a logic gated combination with three measurement signals can be expressed as

OUTPUT=(ΔX)AND(ΔY)AND(ΔZ)

where (ΔX), (ΔY), (ΔZ) may comprise change in heart rate signal from baseline, change in fluid content signal from baseline and change in respiration signal from baseline, and each may have a value of zero or one, based on the values of the signals. For example if the heart rate increase by 10%, (ΔX) can be assigned a value of 1. If fluid content increases by 5%, (ΔY) can be assigned a value of 1. If activity decreases below 10% of a baseline value (ΔZ) can be assigned a value of 1. When each of (ΔX), (ΔY), (ΔZ) is one, the output signal is one, and a flag may be set to trigger an alarm. If any one of (ΔX), (ΔY) or (ΔZ) is zero, the output signal is zero and a flag may be set so as not to trigger an alarm. While a specific example with AND gates has been shown the data can be combined in may ways with known gates for example NAND, NOR, OR, NOT, XOR, XNOR gates. In some embodiments, the gated logic may be embodied in a truth table.

A step 455 sets a flag. The flag can be set in response to the output of the combined signals. In some embodiments, the flag may comprise a binary parameter in which a value of zero does not triggers an alarm and a value of one triggers an alarm.

A step 460 communicates with the patient and/or a health care provider. In some embodiments, the remote site may contact the patient to determine if he or she is okay and communicate the impending decompensation such that the patient can receive needed medical care. In some embodiments, the remote site contacts the health care provider to warn the provider of the impending decompensation and the need for the patient to receive medical care.

A step 465 collects additional measurements. Additional measurements may comprise additional measurements with the at least two signals, for example with greater sampling rates and or frequency of the measurements. Additional measurements may comprise measurements with a additional sensors, for example an onboard microphone to detect at least one of rales, S1 heart sounds, S2 heart sounds, S3 heart sounds, or arrhythmias. In some embodiments, the additional measurements, for example sounds, can be transmitted to the health care provider to diagnose the patient in real time.

The processor system, as described above, can be configured to perform the method 400, including many of the steps described above. It should be appreciated that the specific steps illustrated in FIG. 4A provide a particular method of predicting an impending cardiac decompensation, according to an embodiment of the present invention. Other sequences of steps may also be performed according to alternative embodiments. For example, alternative embodiments of the present invention may perform the steps outlined above in a different order. Moreover, the individual steps illustrated in FIG. 4A may include multiple sub-steps that may be performed in various sequences as appropriate to the individual step. Furthermore, additional steps may be added or removed depending on the particular applications. One of ordinary skill in the art would recognize many variations, modifications, and alternatives.

### Experimental

Clinical studies can be performed on patients to determine and characterize empirically pulmonary edema with an ultrasound device mounted on an adherent device as described above. A suitable number of patients, for example 10 patients comprising normal patients and patients with pulmonary edema may have an adherent device attached to the thorax as described above, and imaging studies such as X-ray and ultrasound can be performed on these patients to determine the amount of edema based on conventional imaging. The amount of edema based on conventional imaging can be correlated with the tissue return signals as described above, so as to determine parameters to determine hydration of tissue based on the return signal. Although reference is made to pulmonary tissue, similar studies can be performed on additional tissues. Additional patients can be studied as needed to determine the empirical correlation of the return signal with tissue hydration, for example 100 patients may be studied.

While the exemplary embodiments have been described in many detail, by way of example and for clarity of understanding, those of skill in the art will recognize that a variety of modifications, adaptations, and changes may be employed. Hence, the scope of the present invention should be limited solely by the appended claims.

1. A method for monitoring a patient having a skin and tissue disposed under the skin, the method comprising:
adhering an adherent device to the skin of the patient, the adherent device comprising an ultrasound transducer and at least one sensor configured to measure patient data;
receiving an ultrasound signal reflected from the tissue; and
determining an amount of fluid of the tissue based on the reflected signal and the patient data measured with the at least one sensor.

2. The method of claim 1 wherein the adherent device is adhered to the skin of the patient at a location corresponding to an accumulation of the fluid in the tissue when the patient is positioned in a fluid sensitive orientation and wherein the sensor measures the fluid sensitive orientation of the patient and the ultrasound signal is transmitted in response to the fluid sensitive orientation of the patient.

3. (canceled)

4. The method of claim 1 wherein the amount of fluid is determined without imaging the tissue.

5-6. (canceled)

7. The method of claim 1 wherein the amount of fluid comprises at least one of a percentage of fluid of the tissue, a relative amount of fluid, a change from a baseline or a percent change of a peak of the signal reflected from the tissue over time.

8. (canceled)

9. The method of claim 1 wherein the signal is configured to measure the amount of fluid from a lung of the patient to determine an amount of fluid disposed in a pleura of the lung.

10-12. (canceled)

13. The method of claim 1 wherein determining the fluid amount disposed in the tissue comprises determining a fluid disposed within a portion of a lung of the patient.

14. (canceled)

15. The method of claim 13 further comprising determining the patient's risk of a cardiac decompensation based on the fluid content within the portion of the lung.

16-18. (canceled)

19. The method of claim 1 further comprising determining an orientation of the patient using the adherent device and transmitting the signal based on the determined orientation.

20. The method of claim 19 wherein transmitting the signal occurs after determining that the orientation of the patient is in a standing or upright position.

21-25. (canceled)

26. An adherent device to measure data from a patient having skin and a tissue disposed beneath the skin, the device comprising:
at least one ultrasonic transducer;
at least one sensor configured to measure patient data;
a support configured to adhere to the skin of the patient to couple the ultrasonic transducer and the at least one sensor to the skin; and
circuitry supported with the support and coupled the ultrasonic transducer and the sensor, the circuitry configured to receive an ultrasound signal reflected from the tissue and the patient data from the at least one sensor,
wherein the support comprises a flexible support configured to stretch with a skin of the patient.

27-34. (canceled)

35. The device of claim 26 wherein the flexible support comprises:
a breathable tape with an adhesive coating;
at least one electrode coupled to the breathable tape and capable of electrically coupling to a skin of the patient;
a printed circuit board connected to the breathable tape to support the printed circuit board with the breathable tape when the tape is adhered to the patient; and
electronic components electrically connected to the printed circuit board and coupled to the at least one electrode to measure physiologic signals of the patient, and coupled to the at least one ultrasonic transducer.

36-44. (canceled)

45. The device of claim 35 wherein the electronic components comprise a processor configured to control the ultrasonic transducer.

46. The device of claim 45 wherein the processor is further configured to control the ultrasonic transducer to receive a reflected portion of the ultrasonic signal.

47. The device of claim 46 wherein the processor is further configured to determine a fluid disposed within a portion of a lung of the patient based on the reflected portion of the ultrasonic signal.

48. The device of claim 47 wherein the processor is configured to determine a fluid disposed within the portion of the lung based on a first reflected portion of the signal compared with a second reflected portion of the signal.

49. The device of claim 46 wherein the processor comprises a processor system configured to determine the patient's risk of cardiac decompensation based on the reflected portion of the ultrasonic signal.

50. (canceled)

51. The device of claim 45 wherein the processor is configured to control the ultrasonic transducer to transmit the ultrasonic signal at predetermined intervals.

52. The device of claim 45 wherein the electric components are coupled to the at least one sensor and the at least one electrode, and wherein the processor is configured to determine breathing cycles of the patient using the at least one sensor, and to transmit the ultrasonic signal based on the breathing cycles.

53. The device of claim 45 wherein the electric components are coupled to at least one sensor and the at least one electrode, and wherein the processor is further configured to determine a sleep status of the patient using the at least one sensor, and to transmit the ultrasonic signal based on the sleep status.

54. The device of claim 45 wherein the electric components are coupled to the at least one sensor and the at least one electrode, and wherein the processor is further configured to determine an orientation of the patient using the at least one sensor, and to transmit the ultrasonic signal based on the orientation.

55-61. (canceled)
